(19) United States
(12) Patent Application Publication
(10) Pub. No.: US 2024/0350441 A1
(43) Pub. Date: Oct. 24, 2024

(54) PHARMACEUTICAL FORMULATION AND A PROCESS FOR ITS PREPARATION

(71) Applicant: SAVA HEALTHCARE LTD

(72) Inventors: Sriram PADMANABHAN, Bangalore, Karnataka (IN); Vinod Ramchandra JADHAV, Dubai (AE)

(21) Appl. No.: 18/254,718

(22) PCT Filed: Mar. 30, 2022
(86) PCT No.: PCT/IB2022/052935

§ 371 Date: May 26, 2023

(30) Foreign Application Priority Data
Dec. 1, 2021 (IN) 202121055757

(51) Int. Cl. A61K 31/192; A61K 9/00; A61K 9/20; A61K 47/10; A61K 47/14; A61K 47/20; A61P 37/04; C07C 51/377

(52) U.S. Cl. CPC A61K 31/192; A61K 9/0019; A61K 9/0075; A61K 9/2013; A61K 47/10; A61K 47/14; A61K 47/20; A61P 37/04; C07C 51/377

(57) ABSTRACT

The present disclosure relates to a pharmaceutical formulation and a process for its preparation. The pharmaceutical formulation comprises cinnamic acid and at least one excipient. The pharmaceutical formulation of the present disclosure has improved patient compliance, and reduced adverse effects. The pharmaceutical formulation of the present disclosure can be used in the treatment of cyclophosphamide induced neutropenia. The present disclosure further relates to a process of preparing the cinnamic acid. The process is simple and economical.

### FIELD

The present disclosure relates to a pharmaceutical formulation and a process for its preparation.

### Definitions

As used in the present disclosure, the following terms are generally intended to have the meaning as set forth below, except to the extent that the context in which they are used indicates otherwise.

MMAD (mass median aerodynamic diameter) refers to the diameter at which 50% of the particles of an aerosol by mass are larger and 50% are smaller.

FPF (Fine-particle fraction) refers to a fraction wherein the fine particle dose is divided by the total emitted dose.

First pass metabolism refers to a phenomenon in which a drug gets metabolized at a specific location in the body that results in a reduced concentration of the active drug upon reaching its site of action or the systemic circulation.

D10 refers to the portion of particles with diameters below the specified value is 10%.

D50 refers to the portion of particles with diameters smaller and larger than a specified value are 50%. Also known as the median diameter.

D90 refers to the portion of particles with diameters below the specified value is 90%.

F-MELT® Type C is a proprietary formulation of carbohydrates, disintegrants and inorganic ingredients, designed as a pre-mix for oral disintegrating tablets (ODTs).

GCSF is Granulocyte colony-stimulating factor (G-CSF or GCSF), also known as colony-stimulating factor 3 (CSF 3) is a glycoprotein that stimulates the bone marrow to produce granulocytes and stem cells and release them into the bloodstream.

FD and C colors are any of the synthetic dyes that in certified batches are permitted for use in foods, drugs, and cosmetics by the Federal Food, Drug, and Cosmetic Act of 1938 and subsequent legislation.

Pure Picroside 1 is an iridoid glycoside which is naturally occurring and obtained from the plant Picrorhiza kurroa.

Picroside 1 with salt is obtained by forced alkali hydroxide degradation of the naturally occurring Picroside 1. The salt remains intact in this degraded form.

Picroside 1 without salt is obtained by forced alkali hydroxide degradation of the naturally occurring Picroside 1, which is cinnamic acid in accordance with the present disclosure. The salt is removed by using the second fluid medium (methanol) in this degraded form.

### BACKGROUND

The background information hereinbelow relates to the present disclosure but is not necessarily prior art.

3-Phenyl-2-propenoic acid, commonly referred to as cinnamic acid is a white, crystalline solid and has a low intensity sweet, honey-like aroma. Cinnamic acid and its derivatives are widely distributed in various fruits, vegetables and flowers. Cinnamic acid is found as both trans-cinnamic acid and cis-cinnamic acid. The more stable isomer is the trans isomer, which occurs naturally and is the usual commercial product. Cinnamic acid acts as antioxidants, an antimicrobial agent, a healing agent, an anti-fungal agent and anti-cancer agent and the like. It is used as a precursor for the synthesis of thermoplastics and flavoring agents, and it is also widely used in cosmetic and health products.

In the current scenario, cancer is the deadliest cause of mortality and very prominent disease across the world, affecting millions of people, majorly in the USA, Europe, and the rest of the world. Currently, there are numerous treatments available including chemotherapy, hormonal therapy, immunotherapy, radiation therapy, surgery, and targeted therapy for the prevention and/or treatment of the malignant cancerous cells. Amongst all, the chemotherapy is more prevalent and frequently used in order to treat cancerous cells. Cyclophosphamide, an alkylating chemotherapeutic agent, is a drug widely applied in the clinic to treat malignant and non-malignant tumors. However, despite its wide spectrum of clinical uses, it exhibits severe cytotoxicity to normal cells both in humans and experimental animals. Its metabolites can interact with the cellular macromolecules such as proteins, membrane lipids, RNA, as well as DNA and induce apoptosis. One of its metabolites, namely acrolein, induces oxidative stress that leads to DNA damage of normal cells and cause toxicities to various organs. One of the worst affected sites is the hematopoietic compartment of bone marrow. Indeed, neutropenia is the most common and frequent adverse effect of cytotoxic chemotherapy. Other treatments are costlier and have serious and/or life-threatening adverse events such as anemia, appetite loss, thrombocytopenia, alopecia, peripheral neuropathy, and the like.

As there is no approved medication for effective management and treatment of cyclophosphamide induced neutropenia, there is room for research and development of the formulation that is safe and has enhanced efficacy.

Therefore, there is felt a need for a pharmaceutical formulation that mitigates the drawbacks mentioned hereinabove.

OBJECTS

Some of the objects of the present disclosure, which at least one embodiment herein satisfies, are as follows.

It is an object of the present disclosure to ameliorate one or more problems of the prior art or to at least provide a useful alternative.

An object of the present disclosure is to provide a pharmaceutical formulation.

Another object of the present disclosure is to provide a pharmaceutical formulation of Cinnamic acid.

Yet another object of the present disclosure is to provide a pharmaceutical formulation of Cinnamic acid that is cost-effective and can be used for all kinds of chemotherapy induced neutropenia.

An object of the present disclosure is to provide a pharmaceutical formulation in the form of dry powder inhalation formulation of Cinnamic acid that avoids the first-pass metabolism.

Another object of the present disclosure is to provide a pharmaceutical formulation in the form of a dry powder inhalation formulation of Cinnamic acid that exerts equivalent/enhanced efficacy at a reduced dose.

Yet another object of the present disclosure is to provide a pharmaceutical formulation of Cinnamic acid in the form of an injectable formulation.

Another object of the present disclosure is to provide a pharmaceutical formulation of Cinnamic acid in the form of an oral formulation.

Yet another object of the present disclosure is to provide a simple process for the preparation of Cinnamic acid.

Other objects and advantages of the present disclosure will be more apparent from the following description, which is not intended to limit the scope of the present disclosure.

### SUMMARY

The present disclosure relates to a pharmaceutical formulation and a process for the preparation of the pharmaceutical formulation.

In an aspect, the pharmaceutical formulation comprises cinnamic acid in an amount in the range of 0.1 mass % to 10 mass % with respect to the total mass of the formulation and at least one excipient is in an amount in the range of 90 mass % to 99.9 mass % with respect to the total mass of the formulation. The cinnamic acid is selected from cis-cinnamic acid and trans-cinnamic acid.

In another aspect, the present disclosure relates to a process for the preparation of a cinnamic acid. The process comprises mixing Picroside 1 with a first fluid medium and an alkali hydroxide is added subsequently under stirring to obtain a mixture. The mixture is maintained at a temperature in the range of 25° C. to 35° C. for a predetermined time period followed by neutralizing the mixture with hydrochloric acid to obtain a neutralized mixture. The fluid medium is removed from the neutralized mixture at a temperature in the range of 40° C. to 50° C. under vacuum to obtain a dry powder comprising a degraded Picroside 1 with salt. The dry powder is mixed with a second fluid medium to obtain a solution comprising a degraded Picroside 1 without salt. The solution is decanted and subsequently evaporating the second fluid medium from the solution at a temperature in the range of 40° C. to 50° C. to obtain the cinnamic acid (the degraded Picroside 1 without salt).

### BRIEF DESCRIPTION OF THE ACCOMPANYING DRAWING

The present disclosure will now be described with the help of the accompanying drawing, in which:

FIG. 1A illustrates a graph depicting the pattern of drug distribution per discharge from stage 2 to 6 of NGI (next generation impactor) in accordance with the present disclosure;

FIG. 1B illustrates a graph depicting the pattern of drug distribution per discharge from the device to MOC (micro-orifice collector, that captures in a collection cup extremely small particles of the drug normally collected on the final filter in other impactors) in accordance with the present disclosure;

FIG. 2 illustrates a graph depicting cumulative (% undersize) particle size distribution of the dry powder inhalation (DPI) formulation of Cinnamic acid and drug distribution in various stages in accordance with the present disclosure;

FIG. 3A illustrates an HPLC chromatogram of pure Picroside 1 in accordance with the present disclosure;

FIG. 3B illustrates an HPLC chromatogram of the degraded Picroside 1 without salt (cinnamic acid of the present disclosure) in accordance with the present disclosure;

FIG. 3C illustrates an HPLC chromatogram of the degraded Picroside 1 with salt in accordance with the present disclosure;

FIG. 3D illustrates a mass chromatogram of pure Picroside 1 and methanol (blank solution) in accordance with the present disclosure;

FIG. 3E illustrates a mass spectrum of the degraded Picroside 1 without salt in accordance with the present disclosure;

FIG. 3F illustrates a FTIR spectrum of the degraded Picroside 1 without salt in accordance with the present disclosure;

FIG. 3G illustrates 1H-NMR spectrum of the degraded Picroside 1 without salt in accordance with the present disclosure;

FIG. 3H illustrates 13C-NMR spectrum of the degraded Picroside 1 without salt in accordance with the present disclosure;

FIG. 3I illustrates a UV spectrum of the degraded Picroside 1 without salt in accordance with the present disclosure;

FIG. 3J illustrates a UV spectrum of the commercial synthetic cinnamic acid in accordance with the present disclosure;

FIG. 4A illustrates the comparative effect of pure Picroside 1 and the degraded Picroside 1 with salt against control and GCSF (granulocyte colony stimulating factor) in Cyclophosphamide induced hematological changes;

FIG. 4B illustrates the comparative effect of pure Picroside 1 and the degraded Picroside 1 with salt against control and GCSF (granulocyte colony stimulating factor) in Cyclophosphamide induced hepatotoxicity;

FIG. 5A illustrates the comparative effect of pure Picroside 1, the degraded Picroside 1 without salt (cinnamic acid of the present disclosure), the commercial synthetic cinnamic acid (0.6 mg/tablet) low dose treated and commercial synthetic cinnamic acid (6 mg/tablet) high dose treated against control and GCSF (granulocyte colony stimulating factor) in Cyclophosphamide induced hematological changes; and

FIG. 5B illustrates the comparative effect of pure Picroside 1, the degraded Picroside 1 without salt (cinnamic acid of the present disclosure), the commercial synthetic cinnamic acid (0.6 mg/tablet) low dose treated and the commercial synthetic cinnamic acid (6 mg/tablet) high dose treated against control and GCSF (granulocyte colony stimulating factor) in Cyclophosphamide induced hepatotoxicity and renal changes.

### DETAILED DESCRIPTION

Embodiments, of the present disclosure, will now be described with reference to the accompanying drawing.

Embodiments are provided so as to thoroughly and fully convey the scope of the present disclosure to the person skilled in the art. Numerous details, are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, well-known processes, well-known apparatus structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure is not to be construed as necessarily requiring their performance as described or illustrated. It is also to be understood that additional or alternative steps may be employed.

Cyclophosphamide, an alkylating chemotherapeutic agent is a drug, widely applied in the clinic to treat malignant and nonmalignant tumors. However, despite its wide spectrum of clinical uses, it exhibits severe cytotoxicity to normal cells both in humans and experimental animals. Its metabolites can interact with the cellular macromolecules such as proteins, membrane lipids, RNA, as well as DNA and induce apoptosis. One of its metabolites, namely acrolein, induces oxidative stress that leads to DNA damage of normal cells and cause toxicities to various organs. One of the worst affected sites is the hematopoietic compartment of bone marrow. Indeed, neutropenia is the most common and frequent adverse effect of cytotoxic chemotherapy. Other treatments are costlier and have serious and/or life-threatening adverse events such as anemia, appetite loss, thrombocytopenia, alopecia, peripheral neuropathy, and the like.

Considering the current situation, absence of any approved medication for effective management and treatment of cyclophosphamide induced neutropenia, there is room to develop a pharmaceutical composition that is safe and efficacious.

The present disclosure provided a pharmaceutical formulation and a process for its preparation.

In an aspect of the present disclosure, the pharmaceutical formulation comprises cinnamic acid as a pharmaceutically active agent in an amount in the range of 0.1 mass % to 10 mass % with respect to the total mass of the formulation and at least one excipient in an amount in the range of 90 mass % to 99.9 mass % with respect to the total mass of the formulation.

The cinnamic acid is selected from trans-cinnamic acid and cis-cinnamic acid.

The excipient is at least one selected from the group consisting of solvent, colouring agent, lubricant, diluent and disintegrant.

In accordance with the present disclosure, the solvent is at least one selected from water, ethanol and isopropyl alcohol. The colouring agent is selected from FD and C colors. The lubricant is at least one selected from magnesium stearate, talc, silica, and stearic acid. The disintegrant is at least one selected from carboxymethylcellulose, and hydroxypropyl methylcellulose. The diluent is selected from polyethylene glycol, dimethyl sulfoxide, ethyl lactate and a combination of D-Mannitol-Xylitol-Micro crystalline Cellulose-Crospovidone-Anhydrous dibasic calcium phosphate mixture.

In an embodiment, the pharmaceutical formulation is in a form selected from oral formulation, injectable formulation, and inhalation formulation, metered dose-inhaler formulation, ointments, gels, patches, ophthalmic formulations, and sprays. In an exemplary embodiment, the pharmaceutical formulation is in the form of an oral formulation. In another exemplary embodiment, the pharmaceutical formulation is in the form of an injectable formulation. In yet another embodiment, the pharmaceutical formulation is in the form of a dry powder inhalation formulation.

In an embodiment, the pharmaceutical formulation is the form of an oral formulation. The oral formulation is in a dosage form selected from the group consisting of a tablet, a capsule, an ointment, a gel, a mouthwash, and suspension. In an exemplary embodiment, the dosage form of the oral pharmaceutical formulation is tablet. The oral formulation comprises cinnamic acid in an amount in the range of 0.1 mass % to 5 mass % with respect to the total mass of the formulation and at least one excipient in an amount in the range of 95 mass % to 99.9 mass % with respect to the total mass of the formulation

In an embodiment, the excipient is at least one selected from the group consisting of solvent, colouring agent, lubricant, diluent, and disintegrant.

In an embodiment, the solvent is at least one selected from the group consisting of water, ethanol and isopropyl alcohol. In an exemplary embodiment, the solvent is water.

In an embodiment, the solvent is in an amount in the range of 10 mass % to 50 mass % with respect to the total mass of the excipients. In an exemplary embodiment, the amount of the solvent is quantity sufficient (in mass %) with respect to the total mass of the excipients.

In an embodiment, the colouring agent is at least one selected from the FD &C colors. In an exemplary embodiment, the colouring agent is selected from Iron Oxide yellow NF, brilliant Blue supra, FD and C Green 3 and sunset yellow.

In an embodiment, the colouring agent is in an amount in the range of 0.5 mass % to 2 mass % with respect to the total mass of the excipients. In an exemplary embodiment, the amount of the colouring agent is 0.7 mass % with respect to the total mass of the excipients.

In an embodiment, the lubricant is at least one selected from the group consisting of magnesium stearate, talc, silica, and stearic acid. In an exemplary embodiment, the lubricant is magnesium stearate.

In an embodiment, the lubricant is in an amount in the range of 0.5 mass % to 2 mass % with respect to the total mass of the excipients. In an exemplary embodiment, the amount of the lubricant is 0.7 mass % with respect to the total mass of the excipients.

In an embodiment, the disintegrant is at least one selected from the group consisting of carboxy methylcellulose, hydroxypropyl methylcellulose, and F melt type C. In an exemplary embodiment, the disintegrant is F melt type C.

In an embodiment, the disintegrant is in an amount in the range of 95 mass % to 98 mass % with respect to the total mass of the excipients. In an exemplary embodiment, the amount of the disintegrant is 96 mass % with respect to the total mass of the excipients.

In an embodiment, the diluent is selected from polyethylene glycol, dimethyl sulfoxide, ethyl lactate and a combination of D-mannitol-xylitol-micro crystalline cellulose-crospovidone-anhydrous dibasic calcium phosphate mixture.

In an embodiment, the total amount of the excipients is in the range of 95 mass % to 99.9 mass % with respect to the total mass of the formulation.

In an embodiment, the pharmaceutical formulation is an injectable formulation. The injectable formulation comprises cinnamic acid in an amount in the range of 1 mass % to 10 mass % with respect to the total mass of the formulation and at least one excipient in an amount in the range of 90 mass % to 99 mass % with respect to the total mass of the formulation.

In an embodiment, at least one excipient is selected from the group consisting of polyethylene glycol, water, ethanol, ethyl lactate, propylene glycol, and dimethyl sulfoxide. In an exemplary embodiment, the excipient is a combination of alcohol and polyethylene glycol, alcohol and water, alcohol and dimethyl sulfoxide, alcohol and ethyl lactate, alcohol and propylene glycol.

In an embodiment, the pharmaceutical formulation is a dry powder inhalation formulation comprises a micronized cinnamic acid, a first lactose, a second lactose and at least one excipient.

In an embodiment, the micronized cinnamic acid has a particle size in the range of 0.1 μm to 10 μm. In an exemplary embodiment, the particle size of the micronized cinnamic acid is 1 to 5 μm.

In an embodiment, the micronized cinnamic acid is in an amount in the range of 1 mass % to 5 mass % with respect to the total mass of the formulation. In an exemplary embodiment, the amount of the micronized cinnamic acid is 2.4 mass %.

In an embodiment, the first lactose has a particle size in the range of 20 μm to 300 μm. In an exemplary embodiment, the particle size of the first lactose is 35 to 190 μm.

In an embodiment, the first lactose is in an amount in the range of 10 mass % to 30 mass % with respect to the total mass of the formulation. In an exemplary embodiment, the amount of the first lactose is 19 mass %.

In an embodiment, the first lactose is respitose SV010.

In an embodiment, the mean particle size (d10) of the first lactose is in the range of 35 μm to 65 μm.

In an embodiment, the mean particle size (d50) of the first lactose is in the range of 95 μm to 125 μm.

In an embodiment, the mean particle size (d90) of the first lactose is in the range of 160 μm to 190 μm.

In an embodiment, the second lactose has a particle size in the range of 0.1 μm to 10 μm. In an exemplary embodiment, the particle size of the second lactose is 0.8 to 7.5 μm.

In an embodiment, the second lactose is in an amount in the range of 75 mass % to 85 mass % with respect to the total mass of the formulation. In an exemplary embodiment, the amount of the second lactose is 78 mass %.

In an embodiment, the second lactose is pharmatose-450 M.

In an embodiment, the mean particle size (d10) of the second lactose is in the range of 0.01 μm μm to 0.1 μm.

In an embodiment, the mean particle size (d50) of the second lactose is in the range of 1 μm to 5 μm.

In an embodiment, the mean particle size (d90) of the second lactose is in the range of 1 μm to 10 μm.

In an embodiment, the mass ratio of the first lactose to the second lactose is in the range of 1:2 to 1:5. In an exemplary embodiment, the mass ratio of the first lactose to the second lactose is 1:4.

In an embodiment, the excipient is selected from magnesium stearate, calcium stearate and zinc stearate. In an exemplary embodiment, the excipient is magnesium stearate.

In an embodiment, the excipient is in an amount in the range of 0.1 mass % to 1 mass % with respect to the total mass of the formulation. In an exemplary embodiment, the amount of the excipient is 0.32 mass %.

In an embodiment, the median mass aerodynamic diameter (MMAD) of the dry powder inhalation formulation is in the range of 2 μm to 5 μm. In an exemplary embodiment, the median mass aerodynamic diameter (MMAD) of the dry powder inhalation formulation is 4.26 μm.

The so obtained dry powder inhalation (DPI) formulation is loaded into size 3 HPMC (Hydroxypropyl methylcellulose) capsules using a capsule filler.

The present disclosure provides the dry powder inhalation formulation that avoids the first-pass metabolism. The formulation of the present disclosure is therapeutically effective at a reduced dose and also observed increased patient compliance.

In an embodiment, the pharmaceutical formulation of cinnamic acid is administered at a dose in the range of 1.5 mg/kg body weight to 30 mg/kg body weight.

In an embodiment, the pharmaceutical formulation has an anti-neutropenic activity.

In another embodiment, the pharmaceutical formulation has an anti-tuberculosis activity, anti-malarial activity and cardiovascular activity.

In an embodiment, the pharmaceutical formulation is further combined with known anti-tuberculosis drugs.

In an embodiment, the pharmaceutical formulation has an anti-viral activity against COVID-19 activity and anti-bacterial activity against bacteria such as Bacillus subtilis and E. coli present in herbal extract powders.

In an embodiment, the pharmaceutical formulation of Cis-cinnamic acid is 120 folds more effective than the trans-form of cinnamic acid.

In an embodiment, the pharmaceutical formulation of Cis-cinnamic acid is in the form of oral formulation, injectable formulation, inhalation formulation, topical formulation, and ophthalmic formulation.

In an embodiment, the pharmaceutical formulation of Cis-cinnamic acid is used in the treatment of neutropenia, tuberculosis, malaria, and cardiovascular diseases.

The present disclosure further provides a method for treating neutropenia, tuberculosis, malaria, viral infection, bacterial infection and cardiovascular diseases in mammals, wherein the method comprises administering the mammal, a therapeutically effective amount of cinnamic acid in an amount in the range of 1.5 mg/kg body weight to 30 mg/kg body weight. In an embodiment of the present disclosure, the mammal is human.

In an embodiment of the present disclosure, the therapeutically effective amount of cinnamic acid is in the range of 15 mg/kg body weight to 25 mg/kg body weight.

In an embodiment of the present disclosure, the pharmaceutical formulation is used for the treatment of neutropenia, tuberculosis, malaria, viral infection, bacterial infection and cardiovascular diseases.

In an embodiment of the present disclosure, the cinnamic acid is used in the treatment of neutropenia, tuberculosis, malaria, viral infection, bacterial infection and cardiovascular diseases, wherein a therapeutically effective amount of the cinnamic acid is in the range of 1.5 mg/kg body weight to 30 mg/kg body weight.

Therapeutically effective amount refers to the amount of a compound that, when administered to a patient, is sufficient to effect such treatment of a particular disease or condition, elicit the desired effect.

In another aspect of the present disclosure, there is provided a process for the preparation of the cinnamic acid.

The process is described in detail.

In a first step, pure Picroside 1 is mixed with a first fluid medium and simultaneously an alkali hydroxide is added under stirring to obtain a mixture.

In an embodiment, the first fluid medium is water.

In an embodiment, the alkali is sodium hydroxide, potassium hydroxide and calcium hydroxide.

In an embodiment, a ratio of the Picroside 1 to the alkali hydroxide is 0.01:50.

In a second step, the mixture is maintained at a temperature in the range of 25° C. to 35° C. for a predetermined time period followed by neutralizing the mixture with hydrochloric acid to obtain a neutralized mixture.

In an embodiment, the predetermined time period is in the range of 30 minutes to 2 hours. In an exemplary embodiment, the predetermined time period is 1 hour.

In a third step, the fluid medium is removed from the neutralized mixture at a temperature in the range of 40° C. to 50° C. under vacuum to obtain a dry powder comprising a degraded Picroside 1 with salt.

In a fourth step, the dry powder is mixed with a second fluid medium to obtain a solution comprising a degraded Picroside 1 without salt. In an embodiment, the salt so obtained (NaCl) is settled at the bottom (a settled salt).

In an embodiment, the second fluid medium is methanol, ethanol, propanol and butanol. In a fifth step, the solution is decanted and subsequently evaporating the second fluid medium from the solution at a temperature in the range of 40° C. to 50° C. to obtain the cinnamic acid (the degraded Picroside 1 without salt).

In an embodiment, after decantation, the settled salt is further washed with the second fluid medium and the washed second fluid medium is evaporated to obtain the cinnamic acid. The cinnamic acid in an amount in the range of 0.1 mass % to 10 mass % prepared in accordance with the present disclosure is formulated with at least one excipient in an amount in the range of 90 mass % to 99.9 mass % for the treatment of neutropenia, tuberculosis, malaria, viral infection, bacterial infection and cardiovascular diseases, wherein the mass percentage of each ingredient is with respect to the total mass of cinnamic acid and excipient.

The foregoing description of the embodiments has been provided for purposes of illustration and is not intended to limit the scope of the present disclosure. Individual components of a particular embodiment are generally not limited to that particular embodiment, but, are interchangeable. Such variations are not to be regarded as a departure from the present disclosure, and all such modifications are considered to be within the scope of the present disclosure.

The present disclosure is further described in light of the following experiments which are set forth for illustration purpose only and not to be construed for limiting the scope of the disclosure. The following experiments can be scaled up to industrial/commercial scale and the results obtained can be extrapolated to industrial scale.

### EXPERIMENTAL DETAILS

### Example 1: Preparation of Pharmaceutical Formulation of Cinnamic Acid in the Form of a Tablet by Using Wet Granulation

The pharmaceutical formulation of cinnamic acid in the form of a tablet was prepared by using wet granulation. The process comprises following steps; 2 mg/tablet of cinnamic acid was dissolved in 5 gm of water to obtain a drug solution. 67.5 mg/tablet of F melt type C and 0.5 mg/tablet of blue color were shifted through sieve no. 40 and 80 and mixed for 5 minutes in a polybag to obtain a blend. The blend of F melt type C, blue color and drug solution was transferred to the wet granulator to obtain the granules of cinnamic acid. Granules were dried in the oven at 60° C. for 15 mins to obtain the dried granules. The dried granules were shifted through sieve no. 40 and charged into a blender to obtain the powder. 0.5 mg/tablet of magnesium stearate was added into the powder and mixed for 5 mins in the polybag and transferred to the compressor to obtain the uncoated tablet of cinnamic acid. The uncoated tablet of the cinnamic acid was characterized by evaluating various tablet parameters as given in table 2

TABLE 1

| | | Experiment No. 1 |
|---|---|---|
| Sr. No. | Ingredients | (in mg/tab) |
| 1. | Cinnamic acid (degraded Picroside 1 | 2 |
| | without salt (cinnamic acid of the | |
| | present disclosure)) | |
| 2. | F melt type C | 67.5 |
| 3. | Blue Color | 0.5 |
| 4. | Water | q.s |
| 5. | Magnesium stearate (lubricant) | 0.5 |

Total weight of the uncoated tablet = 70.50

### F-Melt® Type C is a Combination of D-Mannitol-Xylitol-Micro Crystalline

### Cellulose-Crospovidone-Anhydrous Dibasic Calcium Phosphate Mixture

TABLE 2

| | | | Disintegration |
|---|---|---|---|
| Appearance | Weight | Hardness | time |
| Blue color uncoated | 69.0 mg-73.0 mg | 25-50 N | 10 sec to |
| tablet, round, plain | | | 50 sec |
| on both sides | | | |

### Example 2: Comparative Formulations

The comparative formulations were prepared in the similar manner as disclosed in Example 1, by varying the concentration of ingredients according to the formulations as illustrated in Table 3 below:

### Example 3A: Preparation of Pharmaceutical Formulation of Cinnamic Acid (6 mg/ml) in an Injectable Form by Using Commercial Synthetic Cinnamic Acid

6 mg of cinnamic acid was dissolved in 300 μl of 100% ethanol, sonicated for 5 min and then diluted to 1 ml with 5% PEG 400/5% DMSO/5% ethyl lactate to obtain a clear solution of cinnamic acid in the injectable form (injectable formulation). The formulation was evaluated for stability as per ICH guidelines. The stability parameters of the injectable formulation of Cinnamic acid (6 mg/ml) are tabulated below in table 4a and 4b.

| | | The | The | |
|---|---|---|---|---|
| | | commercial | commercial | |
| | | synthetic | synthetic | |
| | | cinnamic | cinnamic | |
| | | acid 0.6 | acid 6 | |
| | | mg/tablet | mg/tablet | |
| | Pure | low dose | high dose | Placebo |
| | Picroside 1 | treated | treated | group |
| Ingredients | mg/tab | mg/tab | mg/tab | Mg/tab |
| Pure | 2.00 | NA | NA | NA |
| Picroside 1 | | | | |
| The commer- | NA | 0.6 | 6.00 | NA |
| cial synthetic | | | | |
| Cinnamic acid | | | | |
| F melt type C | 67.5 | 68.9 | 63.5 | 69.3 |
| Color | NA | 0.50 | 0.50 | 0.2 |
| Ethanol | q.s | q.s | q.s | q.s |
| Magnesium | 0.5 | 0.5 | 0.50 | 0.5 |
| Stearate | | | | |
| Total weight | 70.00 | 70.50 | 70.50 | 70.00 |
| Pure | 2.00 | NA | NA | NA |
| Picroside 1 | | | | |
| The commer- | NA | 0.6 | 6.00 | NA |
| cial synthetic | | | | |
| Cinnamic acid | | | | |
| F melt type C | 67.5 | 68.9 | 63.5 | 69.3 |
| Color | NA | 0.50 | 0.50 | 0.2 |
| Ethanol | q.s | q.s | q.s | q.s |
| Magnesium | 0.5 | 0.5 | 0.50 | 0.5 |
| Stearate | | | | |
| Total weight | 70.00 | 70.50 | 70.50 | 70.00 |

TABLE 4a
Commercial synthetic Cinnamic acid
(Conc. 6 mg/mL)_Zero day study data

| | | 30% | 30% | 30% Ethanol |
|---|---|---|---|---|
| | | Ethanol | Ethanol | with 5% |
| | 100% | with 3.5% | with 5% | Ethyl |
| | Methanol | PEG400 | DMSO | lactate |
| HPLC | 3379040 | 3128043 | 3276296 | 3175284 |
| area | | | | |
| % solu- | 100% | 92.57% | 96.95% | 93.97% |
| bility | | | | |

TABLE 4b
Commercial synthetic Cinnamic acid
(Conc. 6 mg/mL)_7 days study data

| | | 25° C. | 30° C. | 40° C. |
|---|---|---|---|---|
| | 2-8° C. | 60% RH | 75% RH | 75% RH |
| 30% Ethanol | 100% | 100% | 100% | 100% |
| with 3.5% PEG | | | | |
| 400 % Solubility | | | | |
| 30% Ethanol | 100% | 100% | 100% | 100% |
| with 5% PEG | | | | |
| 400 % Solubility | | | | |
| 30% Ethanol | 100% | 100% | 100% | 100% |
| with 5% DMSO % | | | | |
| Solubility | | | | |
| 30% Ethanol | 100% | 94.41% | 96.94% | 95.50% |
| with 5% Ethyl | | | | |
| lactate % | | | | |
| Solubility | | | | |

Inference: It is evident from table 4a and 4b that formulation of 6 mg/ml is stable in all the diluents tested for the stability. The presence of 30% ethanol ensures that the cinnamic acid of the present disclosure (degraded Picroside 1 without salt) remains in soluble form.

### Example 3B: Preparation of Pharmaceutical Formulation of the Commercial Synthetic Cinnamic Acid (2 mg/ml) in an Injectable Form

2 mg of cinnamic acid was dissolved in 300 μl of 100% ethanol, sonicated for 5 min and then diluted to 1 ml with 5% PEG 400/5% DMSO/5% ethyl lactate/1.4% propylene glycol/water to obtain the clear solution of the commercial synthetic cinnamic acid (2 mg/ml). The formulation was evaluated for stability as per ICH guidelines. The stability parameters of the injectable formulation of Cinnamic acid (2 mg/ml) are tabulated below in table 4c and 4d.

TABLE 4c

| | The commercial synthetic Cinnamic acid (Conc. 2 mg/mL)_7 days study data | | | | | |
|---|---|---|---|---|---|---|
| | | | | 30% Ethanol | 30% Ethanol | |
| | | 30% Ethanol | 30% Ethanol | with 3.5% | with 0.98% | 30% Ethanol |
| | 100% | with 3.5% | with 3.5% | Ethyl | Propyelne | with 70% |
| | Methanol | PEG400 | DMSO | lactate | glycol | Water |
| % solubility | 100% | 99.12% | 89.45% | 100% | 100% | 100% |

TABLE 4d

| | The commercial synthetic Cinnamic acid | | | |
|---|---|---|---|---|
| Conc. Of | (Conc. 2 mg/mL)_7 days study data | | | |
| solvents | | 25° C. | 30° C. | 40° C. |
| (per mL) | 2-8° C. | 60% RH | 75% RH | 75% RH |
| 30% Ethanol | 100% | 100% | 96.49% | 91.44% |
| with 3.5% PEG | | | | |
| 400 % Solubility | | | | |
| 30% Ethanol | 100% | 100% | 95.53% | 100.% |
| with 3.5% DMSO % | | | | |
| Solubility | | | | |
| 30% Ethanol | 100% | 99.06% | 100% | 100% |
| with 3.5% Ethyl | | | | |
| lactate % | | | | |
| Solubility | | | | |
| 30% Ethanol | 100% | 100% | 100% | 100% |
| with 0.98% | | | | |
| Propylene | | | | |
| glycol % | | | | |
| Solubility | | | | |
| 30% Ethanol | 100% | 100% | 100% | 100% |
| with 70% Water % | | | | |
| Solubility | | | | |

Inference: It is evident from 4c and 4d that injectable formulation of 2 mg/ml cinnamic acid is stable in all the diluents tested for the stability. The presence of 30% ethanol ensures that the cinnamic acid remains in soluble state in the solution.

### Example 4: Preparation of Pharmaceutical Formulation of Cinnamic Acid in Dry Powder Inhalation Form

### Dry Mixing:

0.6 gms of the micronized cinnamic acid, 4.76 gms of Respitose SV010 (first lactose) and 19.56 gms of Pharmatose 450 M (Inhalation grade lactose (second lactose)) were mixed using double lined polybags and sifted using 80 mesh size to obtain a drug-lactose blend. Separately, 0.08 gms of magnesium stearate was mixed and added to the drug-lactose blend to obtain a mixture and the mixture was blended at a speed of 15 rpm for 30 minutes to obtain the dry powder inhalation (DPI) formulation of cinnamic acid. The so obtained dry powder inhalation (DPI) formulation of cinnamic acid was loaded into size 3 HPMC capsules using a capsule filler.

### Example 5: In-Vitro Aerodynamic Particle Size Distribution of Cinnamic Acid

The Aerodynamic Particle Size Distribution (APSD) or aerosolization performance is a critical quality attribute for the in vitro characterization of orally inhaled drug products (OINDPs).

The APSD of an aerosol determines the portion of DPI particles that deposit in the body especially in the lower respiratory tract. The particles in the range of 1 to 5 microns reach the lower respiratory tract are considered effective, particles with larger than 5 microns will remain in the upper respiratory tract and are likely to impact the oropharynx and be swallowed, particles smaller than 1 micron will be cleared by lungs clearance mechanism.

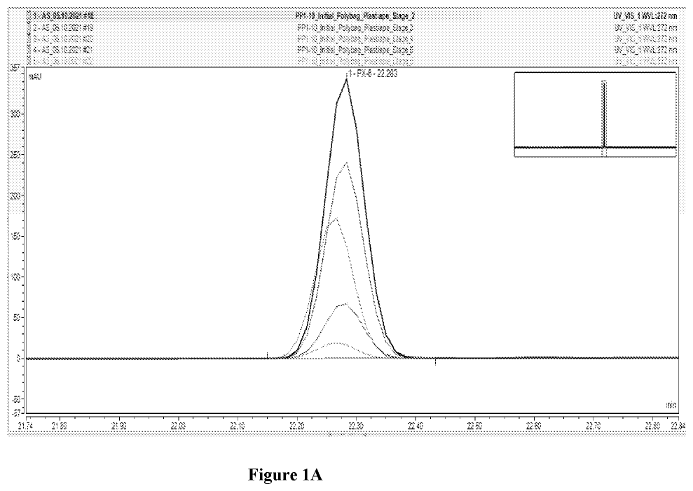

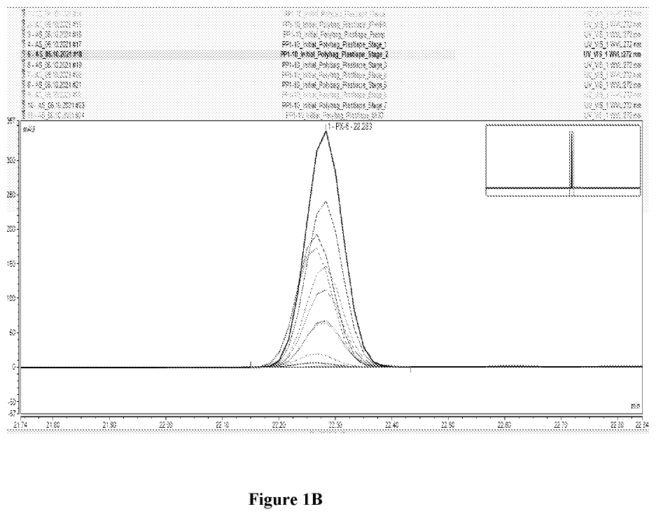

25 mg of Cinnamic acid Dry powder inhalation (DPI) formulation was evaluated for Aerodynamic Particle Size Distribution (APSD). The APSD from each dose of the dry powder inhalation formulation of the present disclosure was evaluated by using Next Generation Impactor (NGI). Dry powder inhalation (DPI) formulation of Cinnamic acid was placed in the inhaler for use and the mouthpiece adapter was attached to the induction port. The pump was switched on at a pressure of 4 kPa pressure drop across the device. The discharge sequence was repeated four times to ensure complete discharge of the powder to the NGI port. After aerosolization, the amount of drug retained in the inhaler device, induction port, mouth-piece adaptor, pre-separator and NGI cups was extracted by washing with a suitable volume of 90:10 methanol:water for quantitative HPLC analysis of cinnamic acid. All the samples were filtered through a 0.45 μm filter and analyzed for cinnamic acid content by HPLC. The important NGI parameters, such as mass median aerodynamic diameter (MMAD), Geometric standard deviation (GSD), the emitted dose (ED) and fine particle fraction (FPF) were calculated using the CITDAS software (COPLEY Scientific, UK). The cinnamic acid DPI, the mean FPF (≤5 μm) was nearly 35% of the nominal dose (which refers to the content of the capsule) for cinnamic acid while the mass median aerodynamic diameter (MMAD) and the GSD value was 4.2 μm and 2.1 respectively. The total emitted dose of active ingredient was 11.081 mg. The pattern of drug distribution per discharge of various stages and device to MOC of NGI illustrated in FIGS. 1A and 1B.

### Example 6: In-Vivo Lung Deposition of Cinnamic Acid

In vivo lung deposition of the dose was evaluated by using a Next Generation Impactor (NGI) for the Cinnamic acid DPI. Each capsule contained 0.6 mg of cinnamic acid of the present disclosure (degraded Picroside 1 without salt) and characterized using Plastiape device with the device at a flow rate of 68 L/min (≈4 KPa).

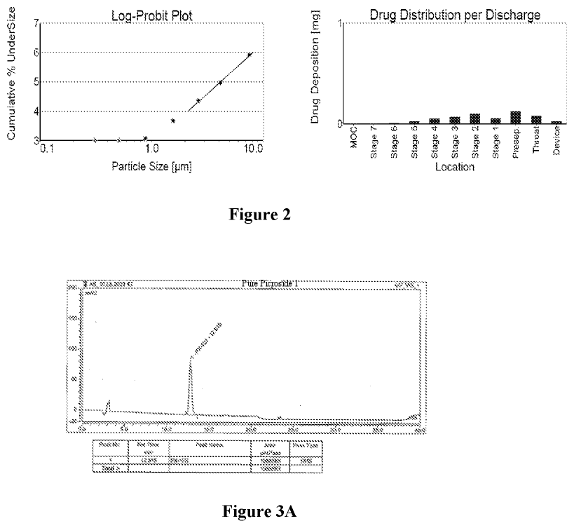

It is evident from FIG. 2 which represents the cumulative (% undersize) particle size distribution of the dry powder inhalation (DPI) formulation of Cinnamic acid as per the present disclosure and the dose of the cinnamic acid DPI was distributed in the different stages of NGI (next generation impactor) and the fine particle dose (respirable dose) and fine particle fraction (respirable fraction) was 0.48 mg and 40.6% respectively while the mean delivered dose was 0.497 mg per actuation. These results meet the requirements of Ph. Eur. 68 that suggests +/−25% of the target dose from a DPI be delivered while that of the USP69 to deliver +/−15% of the target dose.

### Example 7: Alkali Degradation of Pure Picroside 1

180 mg of 95% pure Picroside 1 was mixed in 650 ml of water followed by adding 120 ml of 0.1 NaOH under stirring to obtain a mixture. The so obtained mixture was kept at room temperature for 1 hr followed by neutralizing the mixture with hydrochloric acid to obtain the neutralized mixture having pH 7. The fluid medium was removed from the neutralized mixture at a temperature in the range of 40° C. to 50° C. under vacuum to obtain a dry powder comprising a degraded Picroside 1 with salt. The dry powder was mixed with a second fluid medium to obtain a solution comprising a degraded Picroside 1 without salt. The solution was decanted and subsequently evaporating the second fluid medium from the solution at a temperature in the range of 40° C. to 50° C. to obtain the cinnamic acid of the present disclosure (the degraded Picroside 1 without salt).

The process of the preparation of the cinnamic acid by alkali degradation of pure Picroside 1 is depicted below in scheme 1

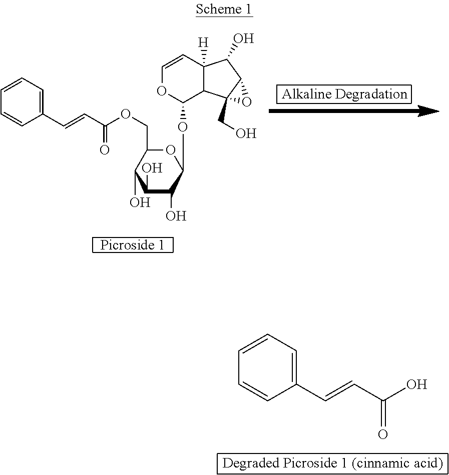

Picroside 1 with salt is obtained by forced alkali hydroxide degradation of the naturally occurring Picroside 1. The salt remains intact in this degraded form.

Picroside 1 without salt is obtained by forced alkali hydroxide degradation of the naturally occurring Picroside 1, which is cinnamic acid in accordance with the present disclosure. The salt is removed by using the second fluid medium (methanol) in this degraded form.

### Example 8: Characterization of the Degraded Picroside 1 without Salt (Cinnamic Acid of the Present Disclosure)

The degraded Picroside 1 without salt without salt (cinnamic acid of the present disclosure) was run on HPLC for evaluating the chromatograms and the dried sample of the degraded Picroside 1 without salt (cinnamic acid of the present disclosure) was also run on the LCMS, NMR and FTIR for evaluating the structure.

HPLC Conditions for the Degraded Picroside 1 without Salt (Cinnamic Acid of the Present Disclosure):

### Mobile Phase:

A) 0.1% Orthophosphoric acid
B) Acetonitrile
Diluent: Methanol
Sample preparation: 10 mg of sample was dissolved in 10 ml of methanol.

### Chromatographic Condition:

Column: BDS Hypersil C18 150×4.6 mm×5 μm.
Column Temp.: 30° C.
Wave length: 255 nm
Flow rate: 1.0 mL/min
Injection Volume: 20 μL
Run time: 40 minutes
Retention Time: At about 21 minutes for Picroside-I degradation
Elution: Gradient

### Gradient Program:

TABLE 5

| Time | % Mobile | % Mobile |
|---|---|---|
| (minutes) | phase A | phase B |
| 0.01 | 80 | 20 |
| 17.00 | 80 | 20 |
| 20.00 | 20 | 80 |
| 32.00 | 20 | 80 |
| 35.00 | 80 | 20 |
| 40.00 | 80 | 20 |

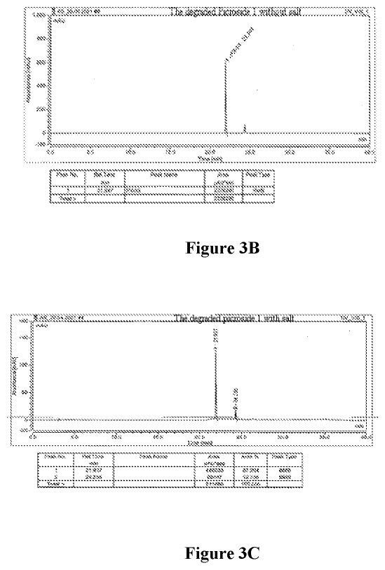

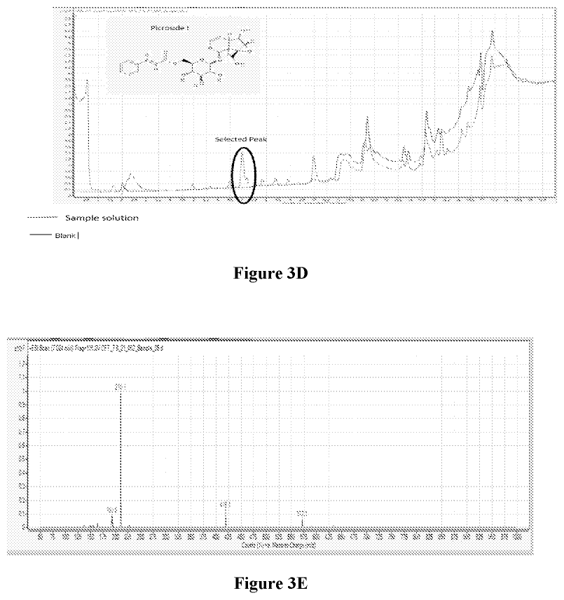

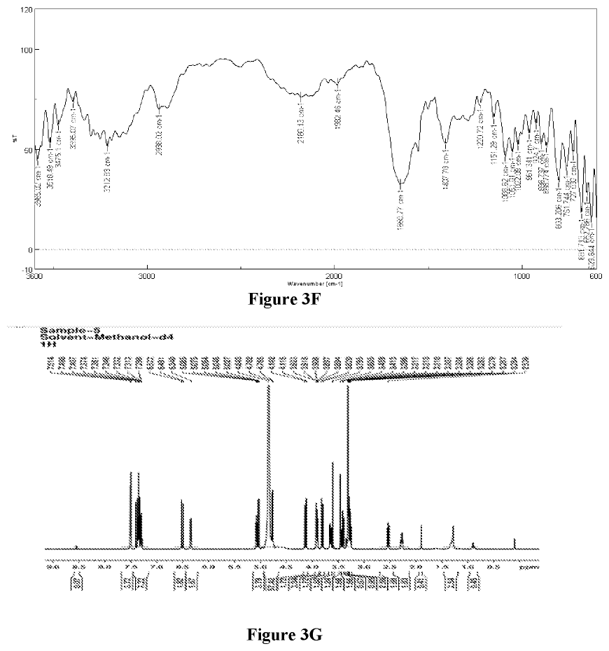

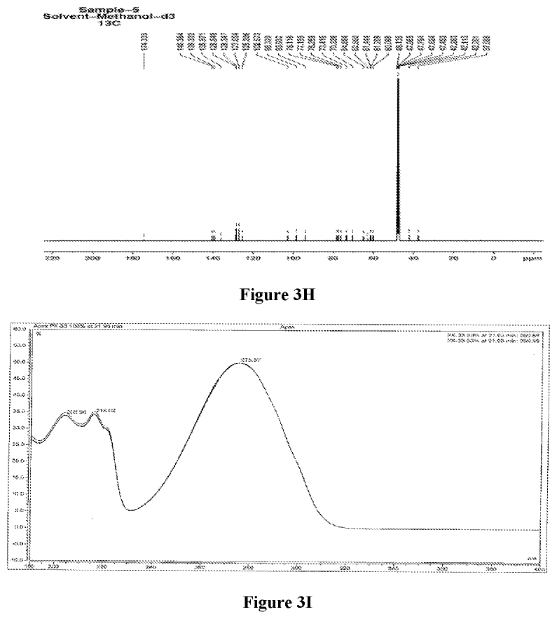

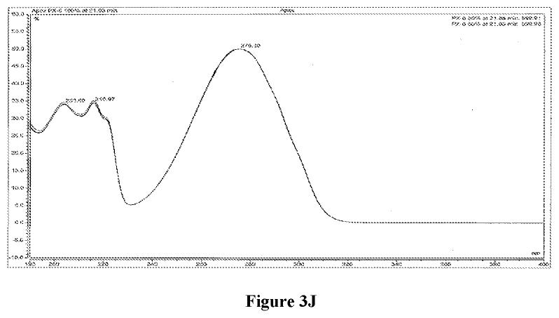

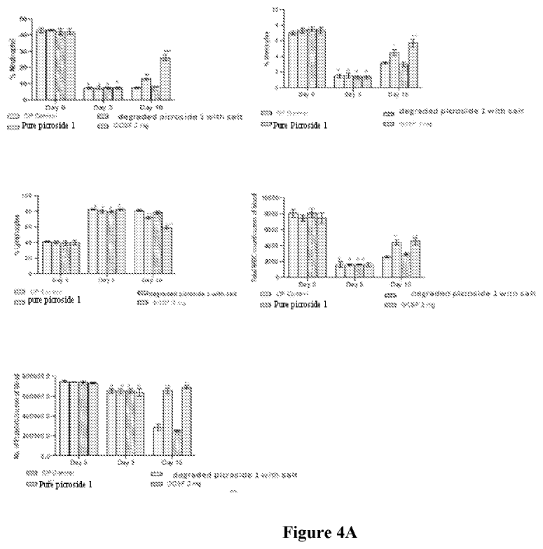

It is evident from FIGS. 3A and 3B that the retention time of the pure Picroside 1 was found to be 12.815 whereas, the retention time of the degraded Picroside 1 without salt (cinnamic acid of the present disclosure) was found to be 21.947.

### LCMS (Liquid Chromatography-Mass Chromatography) Study:

### Mobile Phase:

A: is 5 mM ammonium formate+0.1% formic acid in water,
B: 5 mM ammonium formate+0.1% formic acid in methanol.

Ammonium formate was used in the mobile phase of the LC-MS run, which forms adduct with the degraded Picroside 1 without salt (cinnamic acid of the present disclosure) and shows a molecular mass of 210. Subtracting the mass of observed peak at 210 m/z, the mass of the degraded Picroside 1 without salt (cinnamic acid of the present disclosure) without the adduct is 210−63=147 daltons.

It is clearly evident from the LCMS study and FIGS. 3D and 3E that the degraded Picroside 1 without salt is cinnamic acid having molecular weight of 148 g/mol.

### FTIR (Fourier-Transform Infrared Spectroscopy) Study:

The FTIR results of the degraded Picroside 1 without salt (cinnamic acid of the present disclosure) were tabulated below in table 6 and FIG. 3F.

TABLE 6

| Sr. No | Standard Values | Obtained Values |
|---|---|---|
| 1 | 3300-3400 (OH stretch) | 3395.07 |
| 2 | 3265-3335 (alkynes) | 3212.83 |
| 3 | 1665-1685 (Ketones) | 1650.77 |
| 4 | 1450-1470 (CH bend alkyl) | 1407.78 |
| 5 | 1000-1350 (alkyl halides) | 1220.72 |

NMR Study of the Degraded Picroside 1 without Salt (Cinnamic Acid of the Present Disclosure):

It is evident from FIGS. 3G and 3H that the degraded Picroside 1 without salt showed less signals of the hydrogen and carbon due to the presence of sugars. The NMR signals of the synthetic pure cinnamic acid matches with the peaks observed in the degraded Picroside 1 (without salt). The additional NMR peaks observed in the degraded Picroside 1 (without salt) is due to presence of sugars, which does not affect the identity of the degraded Picroside 1 as cinnamic acid of the present disclosure.

### Comparison of 1H MNR Data

| | Reported peak centers | Peak centers value |
|---|---|---|
| | value for synthetic | for degraded Picroside |
| Sr. no | cinnamic acid (ppm) | 1 (without salt) (ppm) |
| 1 | 7.47 | 7.49 |
| 2 | 7.40 | 7.40 |
| 3 | 7.38 | 7.37 |
| 4 | 6.54 | 6.52 |
| 5 | 6.50 | 6.49 |

Comparison of 13° C. MNR data

| | Reported peak centers | Peak centers value |
|---|---|---|
| | value for synthetic | for degrade Picroside |
| Sr. no | cinnamic acid (ppm) | 1 without salt (ppm) |
| 1 | 128.52 | 128.54 |
| 2 | 128.33 | 128.34 |
| 3 | 135.96 | 135.97 |
| 4 | 140.38 | 140.36 |
| 5 | 174.30 | 174.33 |

Conclusion: It is evident from the HPLC, LCMS, FTIR, NMR (illustrated in FIGS. 3A to 3H) and UV spectroscopy (illustrated in FIGS. 3I and 3J) data that the degraded Picroside 1 without salt is cinnamic acid as the degraded Picroside 1 without salt is showing the similar retention time and similar wavelength similar to the cinnamic acid.

### Example 9: Evaluation of Efficacy of Pure Picroside 1 and the Degraded Picroside 1 with Salt on Cyclophosphamide Induced Neutropenia

### Objectives

1. To induce neutropenia in mice by administration of cyclophosphamide.
2. To determine the effect of treatments of pure Picroside 1 and the degraded Picroside 1 with salt on total WBC count, neutrophils, monocyte, lymphocytes and platelet count.
3. To compare the effect of pure Picroside 1 and the degraded Picroside 1 with salt standard 2 μg G-CSF.

### Materials and Methods

### Materials

Cyclophosphamide was procured from Sigma Aldrich and G-CSF was procured from marketed formulation of Lupin limited, Lupifil (300). Female Swiss Albino mice weighing between 25-27 g were procured from National Institute of Biosciences, Pune.

### Methods

Female Swiss Albino mice were randomized into different groups based on body weight as follows:
Group 1—cyclophosphamide control (CP control),
Group 2—pure Picroside 1 treated,
Group 3—the degraded Picroside 1 with salt and
Group 4—GCSF (granulocyte colony stimulating factor) treated groups.
On day 0, blood was withdrawn from all animals for basal hematological parameters. The animals of all the groups were injected with 150 mg/kg of cyclophosphamide through intraperitoneal route (i.p.). On Day 3, blood was withdrawn from all animals for total and differential count (neutrophils, lymphocytes, and monocytes). The animals were dosed with one additional dose of 100 mg/kg cyclophosphamide on Day 4. From day 5, the animals were administered with respective treatments through oral route in water as vehicle while group 1 was administered with placebo tablets for 5 consecutive days. On day 10, blood was withdrawn from all animals for Total and differential count (neutrophils, lymphocytes, and monocytes), WBCs, Platelets and AST (aspartate aminotransferase) and ALT (alanine aminotransferase).

### Results

Effect of Pure Picroside 1 and the Degraded Picroside 1 with Salt on % Neutrophil in Cyclophosphamide Induced Neutropenia

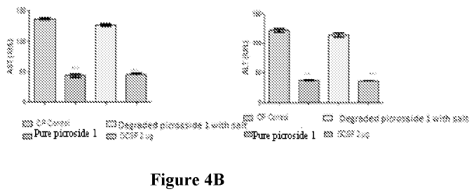

On day 0, the % neutrophil of all the test group of animals was between 42 to 43%, and there was no significant difference observed between the groups on day 0. Administration of cyclophosphamide caused a significant (p<0.001) decrease in the % neutrophil as observed on day 3 with cyclophosphamide control, pure Picroside 1 and the degraded Picroside 1 with salt, and GCSF (granulocyte colony stimulating factor) groups compared to Day 0. Treatment with pure Picroside 1 significantly (p<0.01) alleviated the cyclophosphamide-induced neutropenia, when compared to Placebo control group as tabulated in table 7A and FIG. 4A. However, the degraded Picroside 1 with salt treatment caused no significant change on the cyclophosphamide-induced neutropenia when compared to cyclophosphamide control group of animals. The observed improvement in % neutrophil with pure Picroside 1 was comparable to that of the GCSF (granulocyte colony stimulating factor).

Effect of Pure Picroside 1 and the Degraded Picroside 1 with Salt on % Monocytes in Cyclophosphamide Induced Hematological Changes

On day 0, all test animals had a monocyte percentage of between 7 and 7.5 percent, and there was no significant difference between the groups. Cyclophosphamide administration resulted in a significant (p<0.001) decrease in % monocytes observed on day 3 with the cyclophosphamide control group, pure Picroside 1 and the degraded Picroside 1 with salt, and GCSF (granulocyte colony stimulating factor) groups compared to day 0. A cyclophosphamide-induced decrease in monocyte percentage was significantly reversed by the pure Picroside 1 treatment (p<0.01) when compared to a cyclophosphamide control group as tabulated in table 7A and FIG. 4A. However, the degraded Picroside 1 with salt treatment did not cause any significant change on the cyclophosphamide-induced decrease in % monocyte when compared to cyclophosphamide control group of animals. The observed reversal in pure Picroside 1 was comparable to that of the GCSF (granulocyte colony stimulating factor) treated group. The reversal means that the cyclophosphamide-induced decrease in monocyte percentage was significantly reversed by the pure Picroside 1 treatment. Firstly CP decrease the count of monocyte which further then reversed by pure Picroside 1.

Effect of Pure Picroside 1 and the Degraded Picroside 1 with Salt on % Lymphocytes in Cyclophosphamide Induced Hematological Changes

On day 0, the % lymphocytes of all the test group of animals were between 39 to 41% and there was no significant difference observed between the groups on this day. All groups treated with cyclophosphamide had significantly higher lymphocyte counts on day 3 than day 0 (p<0.01). A significant (p<0.01) reduction in % lymphocytes was observed with the treatment of pure Picroside 1 when compared to the cyclophosphamide control group as tabulated in table 7A and FIG. 4A. However, the degraded Picroside 1 with salt treatment did not cause any significant change on the cyclophosphamide-induced % lymphocyte increase when compared to cyclophosphamide control group of animals. GCSF (granulocyte colony stimulating factor) reversed cyclophosphamide-induced lymphocyte recruitment significantly (p<0.001) compared to the cyclophosphamide control group. The observed reversal in Picroside 1 was comparable to that of the GCSF (granulocyte colony stimulating factor) treated group.

Effect of Pure Picroside 1 and the Degraded Picroside 1 with Salt on Total WBC Count in Cyclophosphamide Induced Hematological Changes

On day 0, the total WBC count of all the test groups of animals was between 7410 to 8085. There was no significant difference observed between the groups on this day. Administration of cyclophosphamide caused a significant (p<0.001) decrease in WBC count observed on day 3 with cyclophosphamide control, pure Picroside 1 and the degraded Picroside 1 with salt, and GCSF (granulocyte colony stimulating factor) groups when compared to day 0. Treatment with pure Picroside 1 and GCSF (granulocyte colony stimulating factor) significantly (p<0.001) reversed the cyclophosphamide induced decrease in WBC count when compared to cyclophosphamide control as tabulated in table 7A and FIG. 4A. However, treatment with the degraded Picroside 1 with salt did not cause a change in cyclophosphamide-induced decreased WBC count compared with the cyclophosphamide control group. The observed reversal in pure Picroside 1 was comparable to that of the GCSF (granulocyte colony stimulating factor) treated group.

Effect of Pure Picroside 1 and the Degraded Picroside 1 with Salt on Platelet Count in Cyclophosphamide Induced Hematological Changes

On day 0, the platelet count of all the test groups of animals was between 732400 to 748400. There was no significant difference observed between the groups on this day. Administration of cyclophosphamide caused a significant (p<0.001) decrease in platelet count as observed on day 3 with cyclophosphamide control, pure Picroside 1 and the degraded Picroside 1 with salt and GCSF (granulocyte colony stimulating factor) groups when compared to day 0. Treatment with Picroside 1 improved the platelet count significantly (p<0.001) to that of the cyclophosphamide control group as tabulated in table 7A and FIG. 4A. However, treatment with the degraded Picroside 1 with salt did not cause any change in platelet count compared with the cyclophosphamide control group. Treatment with GCSF (granulocyte colony stimulating factor) resulted in a significant increase in platelet count as compared to a cyclophosphamide control group. The observed increase in platelet count in pure Picroside 1 group was comparable to that of the GCSF (granulocyte colony stimulating factor) treated group.

TABLE 7A
Effect of pure Picroside 1 and the degraded Picroside 1 with
salt in Cyclophosphamide induced Hematological Changes

| | | Cyclophos- | | | GCSF |
|---|---|---|---|---|---|
| | | phamide | | The degraded | (granulocyte |
| | | control (CP | Pure | Picroside 1 | colony stimulating |
| Parameters | Days | control) | Picroside 1 | with salt | factor) 2 μg |
| % Neutro- | Day 0 | 42.82 ± 1.56 | 43 ± 0.68 | 42.00 ± 1.96 | 42.16 ± 1.74 |
| phils | Day 3 | 7.16 ± 0.70 | 7 ± 0.92 | 7.16 ± 0.74 | 7.16 ± 0.48 |
| | Day 10 | 7.32 ± 0.32 | 12.66 ± 0.66** | 8.00 ± 0.26 | 26.00 ± 1.66*** |
| % | Day 0 | 7.00 ± 0.26 | 7.32 ± 0.32 | 7.50 ± 0.34 | 7.32 ± 0.42 |
| Monocytes | Day 3 | 1.50 ± 0.22 | 1.50 ± 0.34 | 1.32 ± 0.20 | 1.32 ± 0.22 |
| | Day 10 | 3.16 ± 0.16 | 4.50 ± 0.34* | 3.00 ± 0.26 | 5.66 ± 0.48*** |
| % Lym- | Day 0 | 41.32 ± 1.08 | 40.32 ± 1.56 | 39.32 ± 2.82 | 40.00 ± 3.22 |
| phocytes | Day 3 | 82.82 ± 1.08 | 80.00 ± 1.92 | 79.66 ± 1.22 | 82.50 ± 1.40 |
| | Day 10 | 81.32 ± 1.08 | 72.00 ± 1.46** | 78.5 ± 2.16 | 59.32 ± 2.24*** |
| Platelet | Day 0 | 748400 ± 12380 | 741600 ± 3476 | 740500 ± 10260 | 732400 ± 7528 |
| Count(per | Day 3 | 655400 ± 21660 | 647900 ± 26900 | 652100 ± 24380 | 637100 ± 38470 |
| cu · mm | Day 10 | 285200 ± 29920 | 653300 ± 26550*** | 250300 ± 8504 | 690300 ± 15510*** |
| of blood) | | | | | |
| WBC | Day 0 | 8038 ± 521.90 | 7410 ± 414.90 | 8085 ± 564.40 | 7438 ± 626.20 |
| Count(per | Day 3 | 1608 ± 340.30 | 1562 ± 145.90 | 1588 ± 74.22 | 1580 ± 235.00 |
| cu · mm | Day 10 | 2578 ± 99.58 | 4380 ± 333.30*** | 2940 ± 128.50 | 4518 ± 406.00*** |
| of blood) | | | | | |

#p < 0.001 when compared to day 3
*p < 0.05,
**p < 0.01,
***p < 0.001 when compared to Cyclophosphamide control

Inference: It is evident from table 7A and FIG. 4A that administration of Cyclophosphamide caused a significant decrease in % neutrophil, % monocytes, total WBC count, and platelet count and significant increase in % lymphocyte count. Further, treatment with pure Picroside 1 caused alleviation in cyclophosphamide-induced decrease in % neutrophil, % monocytes, total WBC count and also alleviated the increased lymphocyte count. There was an improvement in platelet count in the pure Picroside 1 treated group, and it was statistically significant. Treatment with the degraded Picroside 1 with salt did not significantly change in % neutrophil, % monocytes, total WBC count, and platelet count. However, the observed changes in pure Picroside 1 treatment group were comparable to that of GCSF (granulocyte colony stimulating factor) group, with lesser efficacy.

Effect of Pure Picroside 1 and the Degraded Picroside 1 with Salt on AST (Aspartate Aminotransferase) (IU/L) in Cyclophosphamide Induced Hepatotoxicity

The cyclophosphamide control group showed elevated level of AST (aspartate aminotransferase) after administration of cyclophosphamide. A significant (p<0.001) decrease in AST (aspartate aminotransferase) level was observed following the administration of pure Picroside 1 compared to a cyclophosphamide control group as tabulated in table 7B and FIG. 4B Administration of the degraded Picroside 1 with salt lowers AST (aspartate aminotransferase) significantly (p<0.01) compared to the cyclophosphamide control group. There was a significant (p<0.001) reduction in AST (aspartate aminotransferase) levels of the GCSF (granulocyte colony stimulating factor) treated group when compared to the cyclophosphamide control group.

Effect of Pure Picroside 1 and the Degraded Picroside 1 with Salt on ALT (Alanine Aminotransferase) (IU/L) in Cyclophosphamide Induced Hepatotoxicity

The administration of cyclophosphamide in the cyclophosphamide control group had caused a significant increase in the ALT (alanine aminotransferase)_level. Administration of pure Picroside 1 to the animals caused a significant reversal (p<0.001) of ALT (alanine aminotransferase) level when compared to the cyclophosphamide control group as tabulated in table 7B and FIG. 4B Administration of the degraded Picroside 1 with salt showed no significant change in ALT (alanine aminotransferase) level as compared with the cyclophosphamide control group. The observed decrease in ALT (alanine aminotransferase) in pure Picroside 1 was comparable (p<0.001) to that of ALT (alanine aminotransferase) levels attained upon treatment with GCSF (granulocyte colony stimulating factor).

TABLE 7B
Effect of pure Picroside 1 and the degraded Picroside
1 with salt in Cyclophosphamide induced hepatotoxicity

| | | | | GCSF |
|---|---|---|---|---|
| | | | | (granulocyte |
| | Cyclophosphamide | | The degraded | colony |
| Parameters/ | control | Pure | Picroside 1 | stimulating |
| Groups | (CP control) | Picroside 1 | with salt | factor) 2 μg |
| AST (aspartate | 136.00 ± 1.86 | 43.66 ± 2.68*** | 126.80 ± .2.00* | 45.16 ± 2.72*** |
| aminotransferase) | | | | |
| (IU/L) | | | | |
| ALT (alanine | 121.80 ± 3.40 | 37.32 ± 0.66*** | 113.80 ± 2.96 | 36.16 ± 0.70*** |
| aminotransferase) | | | | |
| (IU/L) | | | | |

***p < 0.001 when compared to CP control

Inference: It is evident from table 7B and FIG. 4B that administration of Cyclophosphamide caused a significant increase in AST (aspartate aminotransferase), and ALT (alanine aminotransferase). Further, there was a significant decrease in the AST (aspartate aminotransferase) and ALT (alanine aminotransferase) levels on treatment with pure Picroside 1.

Hence, it is evident from table 7A, 7B and FIGS. 4A and 4B that the degraded Picroside 1 with salt failed to correct neutropenia condition in animals.

### Example 10: Evaluation of Efficacy of the Commercial Synthetic Cinnamic Acid on Cyclophosphamide Induced Neutropenia

### Objectives

1. To induce neutropenia by administration of cyclophosphamide.
2. To determine the effect of the commercial synthetic cinnamic acid (0.6 mg/tablet)—low dose treated and the commercial synthetic cinnamic acid (6 mg/tablet)—high dose treated on total WBC count, neutrophils, monocyte, lymphocytes, platelet count, liver parameters SGOT (Serum glutamic oxaloacetic transaminase), SGPT (Serum glutamic pyruvic transaminase), Kidney parameters-serum creatinine, blood urea nitrogen and uric acid.
Materials and Methods:

Materials: Female Swiss Albino mice weighing between 25-27 g was procured from Global Bioresearch Solutions Pvt Ltd., Pune.

Methods: Female Swiss Albino mice were randomized into 7 groups of 6 animals each based on body weight as follows:
Group 1—Placebo group (plain water/observational control),
Group 2—cyclophosphamide control (CP control),
Group 3—GCSF (granulocyte colony stimulating factor) 2 μg per animal through s.c. route,
Group 4 the commercial synthetic cinnamic acid (0.6 mg/tablet) low dose treated,
Group 5 the commercial synthetic cinnamic acid (6 mg/tablet) high dose treated,
Group 6 Pure Picroside 1 and
Group 7 the degraded Picroside 1 without salt (cinnamic acid of the present disclosure) treated groups.

On day 0, blood was withdrawn from all animals for basal hematological parameters. All the groups of animals except placebo group were injected with 150 mg/kg of cyclophosphamide through intraperitoneal route (i.p.), while group 1 was injected with water for injection, i.p. On Day 3, blood was withdrawn from all animals for Total and differential count (neutrophils, lymphocytes, and monocytes). The animals of group 2 to 7 were dosed with one additional dose of 100 mg/kg cyclophosphamide on Day 4. From day 5, group 4 and group 5 animals were administered with the commercial synthetic cinnamic acid (0.6 mg/tablet) low dose treated and the commercial synthetic cinnamic acid (6 mg/tablet) high dose treated, while group 6 and group 7 were administered with pure Picroside 1 and the degraded Picroside 1 without salt (cinnamic acid of the present disclosure) for 5 consecutive days. The cyclophosphamide control group was injected with water for injection from day 5 to day 9. On day 10, blood was withdrawn from all animals for total and differential count (neutrophils, lymphocytes, and monocytes), platelets, liver parameters AST (aspartate aminotransferase), ALT (alanine aminotransferase), and renal parameters—serum creatinine, blood urea nitrogen and uric acid.

### Preparation and Administration of Doses:

Pure Picroside 1—1 tablet was dissolved in 0.1 ml of sterile water for injection and 0.1 ml was injected as subcutaneous injection.
The degraded Picroside 1 without salt (cinnamic acid of the present disclosure)—1 tablet was dissolved in 0.1 ml of sterile water for injection and 0.1 ml was injected as subcutaneous injection.
The Cinnamic acid low dose (0.6 mg/tablet) treated tablet was dissolved in 0.1 ml of sterile water for injection and 0.1 ml was injected as subcutaneous injection.
The Cinnamic acid high dose treated (6 mg/tablet) tablet was dissolved in 0.1 ml of sterile water for injection and 0.1 ml was injected as subcutaneous injection.

### Results

Effect of the Commercial Synthetic Cinnamic Acid—Low Dose Treated (0.6 mg/Tablet) and the Commercial Synthetic Cinnamic Acid (6 mg/Tablet)—High Dose Treated on % Neutrophil in Cyclophosphamide Induced Neutropenia

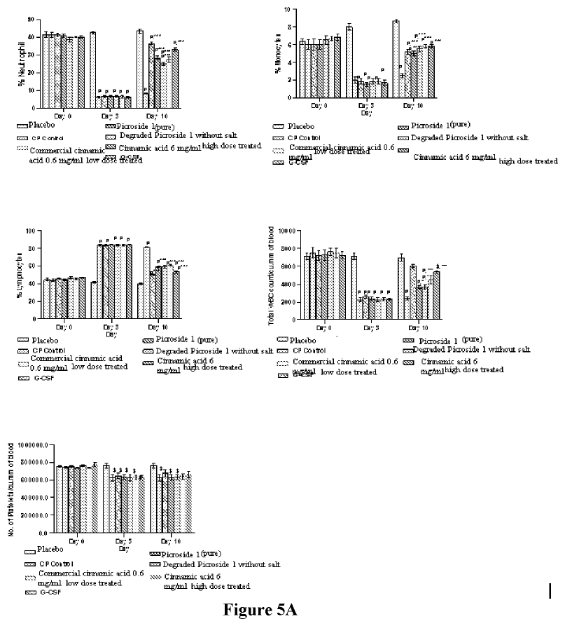

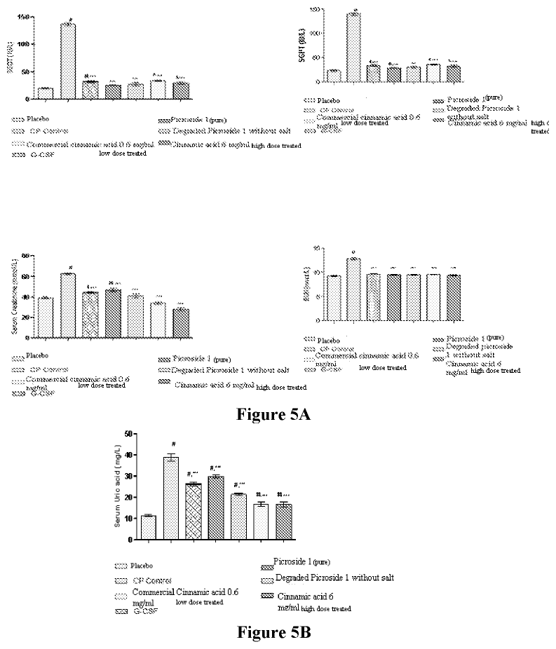

On day 0, the % neutrophil of all the test group of animals were between 38 to 41% and there was no significant difference observed between the groups on day 0. Administration of cyclophosphamide caused a significant (p<0.001) decrease in the % neutrophil as observed on day 3 with all the cyclophosphamide administered groups compared to placebo group. Treatment with the commercial synthetic cinnamic acid (0.6 mg/tablet) low dose treated and the commercial synthetic cinnamic acid (6 mg/tablet) high dose treated significantly (p<0.001) alleviated the cyclophosphamide-induced neutropenia, when compared to CP Control group as tabulated in table 8A and FIG. 5A. This observed improvement in % neutrophil count was superior to that of observed in the commercial synthetic cinnamic acid (0.6 mg/tablet) low dose treated and the commercial synthetic cinnamic acid (6 mg/tablet) high dose treated were comparable to that of the GCSF.

Effect of the Commercial Synthetic Cinnamic Acid (0.6 mg/Tablet) Low Dose Treated and the Commercial Synthetic Cinnamic Acid (6 mg/Tablet) High Dose Treated on % Monocytes in Cyclophosphamide Induced Hematological Changes

On day 0, all test animals had a monocyte percentage of between 6 and 6.82 percent, and there was no significant difference between the groups. Cyclophosphamide administration resulted in a significant (p<0.001) decrease in % monocytes observed on day 3 with all the cyclophosphamide administered groups compared to placebo group. Cyclophosphamide-induced decrease in monocyte percentage was significantly (p<0.001) reversed by the commercial synthetic cinnamic acid (0.6 mg/tablet) low dose treated and the commercial synthetic cinnamic acid (6 mg/tablet) high dose treated and GCSF (granulocyte colony stimulating factor) treatments when compared to a cyclophosphamide control group as tabulated in table 8A and FIG. 5A. The observed reversal in the commercial synthetic cinnamic acid (0.6 mg/tablet) low dose treated and the commercial synthetic cinnamic acid (6 mg/tablet) low dose treated were better than pure Picroside 1, degraded Picroside 1 without salt (cinnamic acid of the present disclosure) and comparable to that of the GCSF (granulocyte colony stimulating factor) treated group.

Effect of the Commercial Synthetic Cinnamic Acid (0.6 mg/Tablet) Low Dose Treated and the Commercial Synthetic Cinnamic Acid (6 mg/Tablet) High Dose Treated on % Lymphocytes in Cyclophosphamide Induced Hematological Changes

On day 0, the % lymphocytes of all the test group of animals were between 43 to 46% and there was no significant difference observed between the groups on this day. All groups treated with cyclophosphamide had significantly (p<0.001) higher lymphocyte counts when compared to placebo group. A significant (p<0.001) decrease in % lymphocytes was observed with the treatment of the commercial synthetic cinnamic acid (0.6 mg/tablet) low dose treated and the commercial synthetic cinnamic acid (6 mg/tablet) low dose treated and GCSF (granulocyte colony stimulating factor) when compared to the CP control (cyclophosphamide) group as tabulated in table 8A and FIG. 5A. GCSF (granulocyte colony stimulating factor) reversed cyclophosphamide-induced lymphocyte recruitment significantly (p<0.001) compared to the CP control group. The observed reversal in the commercial synthetic cinnamic acid (0.6 mg/tablet) low dose treated and the commercial synthetic cinnamic acid (6 mg/tablet) high dose treated were better than pure Picroside 1, degraded Picroside 1 without salt (cinnamic acid of the present disclosure) and comparable to that of the GCSF (granulocyte colony stimulating factor) treated group.

Effect of the Commercial Synthetic Cinnamic Acid (0.6 mg/Tablet) Low Dose Treated and the Commercial Synthetic Cinnamic Acid (6 mg/Tablet) High Dose Treated on Total WBC Count in Cyclophosphamide Induced Hematological Changes

On day 0, the total WBC count of all the test groups of animals was between 7139 to 7644. There was no significant difference observed between the groups on this day. Administration of cyclophosphamide caused a significant (p<0.001) decrease in WBC count observed on day 3 with all cyclophosphamide administered groups when compared to placebo group. Treatment with the commercial synthetic cinnamic acid (0.6 mg/tablet) low dose treated and the commercial synthetic cinnamic acid (6 mg/tablet) high dose treated and GCSF (granulocyte colony stimulating factor) significantly (p<0.001) reversed the cyclophosphamide induced decrease in WBC count when compared to cyclophosphamide control as tabulated in table 8A and FIG. 5A. The observed reversal in the commercial synthetic cinnamic acid (0.6 mg/tablet) low dose treated and the commercial synthetic cinnamic acid (6 mg/tablet) high dose treated was comparable to that of the GCSF (granulocyte colony stimulating factor) treated group and was better than pure Picroside 1, the degraded Picroside 1 without salt (cinnamic acid of the present disclosure).

Effect of the Commercial Synthetic Cinnamic Acid (0.6 mg/Tablet) Low Dose Treated and the Commercial Synthetic Cinnamic Acid (6 mg/Tablet) High Dose Treated on Platelet Count in Cyclophosphamide Induced Hematological Changes

On day 0, the platelet count of all the test groups of animals was between 735700 to 772500. There was no significant difference observed between the groups on this day. Administration of cyclophosphamide caused a significant (p<0.05) decrease in platelet count in all cyclophosphamide treated groups compared to placebo group. Treatment with the commercial synthetic cinnamic acid (0.6 mg/tablet) low dose treated and the commercial synthetic cinnamic acid (6 mg/tablet) high dose treated did not cause significant change in the platelet count on day 10 as tabulated in table 8A and FIG. 5A when compared to cyclophosphamide control; however, there was no significant change in the commercial synthetic cinnamic acid (0.6 mg/tablet) low dose treated and the commercial synthetic cinnamic acid (6 mg/tablet) high dose treated when compared to placebo group also. Treatment with GCSF (granulocyte colony stimulating factor) resulted in a significant (p<0.05) increase in platelet count as compared to a cyclophosphamide control group.

TABLE 8A
Effect of the commercial synthetic cinnamic acid (0.6 mg/tablet) low dose treated and the commercial synthetic cinnamic
acid (6 mg/tablet) high dose treated on hematological parameters in Cyclophosphamide induced Neutropenia

| | | | | | | The | | |
|---|---|---|---|---|---|---|---|---|
| | | | | | | degraded | | |
| | | | | | | Picroside | The | The |
| | | | | G-CSF | | 1without | commercial | commercial |
| | | | | (granulocyte | | salt | synthetic | synthetic |
| | | | | colony | | (cinnamic | Cinnamic | Cinnamic |
| | | | CP | stimulating | Pure | acid of the | acid | acid |
| | | Placebo | Control | factor) | Picroside | present | (0.6 mg/ | (6 mg/ |
| Parameters | Day | group | (cyclophosphamide) | (2 μg) | 1 | disclosure) | tablet) | tablet) |
| % Neutrophils | 0 | 41.67 ± | 41.5 ± | 41.33 ± | 41.00 ± | 38.83 ± | 40.33 ± | 40.17 ± |
| | | 1.43 | 1.455 | 0.9189 | 0.9661 | 1.302 | 0.4216 | 0.7491 |
| | 3 | 42.67 ± | 6.167 ± | 6.667 ± | 6.50 ± | 6.667 ± | 6.50 ± | 6.333 ± |
| | | 0.7149 | 0.4773^{#} | 0.5578^{#} | 0.4282^{#} | 0.4216^{#} | 0.5627^{#} | 0.4944^{#} |
| | 10 | 43.67 ± | 8.167 ± | 36.67 ± | 28.33 ± | 25.17 ± | 27.67 ± | 33.17 ± |
| | | 1.229 | 0.3073^{#} | 0.4944^{#,}*** | 1.202^{#,}*** | 0.9458^{#,}*** | 1.647^{#,}*** | 0.8333^{#,}*** |
| % Monocytes | 0 | 6.33 ± | 6.00 ± | 6.00 ± | 6.00 ± | 6.50 ± | 6.66 ± | 6.82 ± |
| | | 0.3332 | 0.5164 | 0.6325 | 0.5774 | 0.5 | 0.2108 | 0.4014 |
| | 3 | 8.00 ± | 2.00 ± | 1.833 ± | 1.50 ± | 1.82 ± | 1.82 ± | 1.66 ± |
| | | 0.3651 | 0.3651^{#} | 0.3073^{#} | 0.2236^{#} | 0.3073^{#} | 0.3073^{#} | 0.3333^{#} |
| | 10 | 8.66 ± | 2.50 ± | 5.167 ± | 5.00 ± | 5.50 ± | 5.82 ± | 5.82 ± |
| | | 0.2108 | 0.2236^{#} | 0.3073^{#,}*** | 0.2582^{#,}*** | 0.3416^{#,}*** | 0.1667^{#,}*** | 0.1667^{#,}*** |
| % Lymphocytes | 0 | 44.33 ± | 43.83 ± | 45.5 ± | 44.33 ± | 46.5 ± | 45.17 ± | 46.83 ± |
| | | 1.333 | 1.046 | 0.6191 | 0.9545 | 1.408 | 0.7491 | 0.654 |
| | 3 | 41.33 ± | 83.83 ± | 83.5 ± | 84 ± | 83.5 ± | 83.67 ± | 84 ± |
| | | 0.7149 | 0.7923^{#} | 0.7638^{#} | 0.7303^{#} | 0.6191^{#} | 0.9189^{#} | 0.6325^{#} |
| | 10 | 39.67 ± | 81.33 ± | 50.17 ± | 58.67 ± | 58.83 ± | 61 ± | 53 ± |
| | | 1.085 | 0.4944^{#} | 0.654^{#,}*** | 1.085^{#,}*** | 1.833^{#,}*** | 1^{#,}*** | 1.065^{#,}*** |
| Total WBC | 0 | 7139 ± | 7513 ± | 7255 ± | 7301 ± | 7644 ± | 7465 ± | 7237 ± |
| Count | | 398.5 | 581.4 | 556.1 | 587.5 | 429 | 529.2 | 406.1 |
| (per cu · mm | 3 | 7119 ± | 2265 ± | 2617 ± | 2365 ± | 2253 ± | 2289 ± | 2306 ± |
| of blood) | | 349.7 | 187^{#} | 178.9^{#} | 211.1^{#} | 204.7^{#} | 187.9^{#} | 143.4^{#} |
| | 10 | 6969 ± | 2421 ± | 6030 ± | 3674 ± | 3705 ± | 4460 ± | 5389 ± |
| | | 386.3 | 205.9^{#} | 181.9*** | 194.9^{#,}* | 256.2^{#,}* | 459.1^{#,}*** | 130.3^{$$,}*** |
| No of | 0 | 749900 ± | 744000 ± | 752200 ± | 735700 ± | 759400 ± | 736600 ± | 772500 ± |
| Platelets | | 11150 | 10120 | 8805 | 5085 | 9648 | 5754 | 23670 |
| (per cu · mm | 3 | 762300 ± | 625800 ± | 645800 ± | 631700 ± | 624000 ± | 630500 ± | 637000 ± |
| of blood) | | 27840 | 39180^{$} | 30500^{$} | 28330^{$} | 33600^{$} | 25830^{$} | 12590^{$} |
| | 10 | 762200 ± | 626100 ± | 674900 ± | 626800 ± | 631900 ± | 641400 ± | 660300 ± |
| | | 27780 | 39230^{$} | 40260 | 33590^{$} | 25780^{$} | 31940 | 34170 |

^{$}p < 0.05,
^{$$}p < 0.01,
^{#}p < 0.001 when compared to placebo group
*p < 0.01,
***p < 0.001 when compared to CP control

Inference: It is evident from table 8A that the test compounds (pure Picroside 1, the degraded Picroside without salt (cinnamic acid of the present disclosure), the commercial cinnamic acid (0.6 mg/tablet) low dose treated and the commercial cinnamic acid (6 mg/tablet) high dose treated were administered as 1 tablet per animal per day from day 5 for 5 days; CP control was administered with placebo tablet. Further, it is evident from table 8A that pure Picroside 1 and the degraded Picroside 1 without salt (cinnamic acid of the present disclosure) shows better anti-neutropenic activity. The commercial synthetic cinnamic acid (0.6 mg/tablet) low dose treated and the commercial synthetic cinnamic acid (6 mg/tablet) high dose treated also are active to address neutropenia.

Effect of the Commercial Synthetic Cinnamic Acid (0.6 mg/Tablet) Low Dose Treated and the Commercial Synthetic Cinnamic Acid (6 mg/Tablet) High Dose Treated on SGOT (Serum Glutamic Oxaloacetic Transaminase) (IU/L) in Cyclophosphamide Induced Neutropenia

The cyclophosphamide control group showed a significantly (p<0.001) elevated level of SGOT (Serum glutamic oxaloacetic transaminase)_after administration of cyclophosphamide when compared with the placebo group. A significant (p<0.001) decrease in SGOT (Serum glutamic oxaloacetic transaminase)_level was observed following the administration of the commercial synthetic cinnamic acid (0.6 mg/tablet) low dose treated and the commercial synthetic cinnamic acid (6 mg/tablet) high dose treated compared to a cyclophosphamide control group as tabulated in table 8B and FIG. 5B. There was a significant (p<0.001) reduction in SGOT (Serum glutamic oxaloacetic transaminase)_level of the GCSF (granulocyte colony stimulating factor) treated group when compared to the cyclophosphamide control group. However, this decrease was not superior to the decrease observed upon treatment with pure Picroside 1 and the degraded Picroside 1 without salt (cinnamic acid of the present disclosure).

Effect of the Commercial Synthetic Cinnamic Acid (0.6 mg/Tablet) Low Dose Treated and the Commercial Synthetic Cinnamic Acid (6 mg/Tablet) High Dose Treated on SGPT (Serum Glutamic Pyruvic Transaminase) (IU/L) in Cyclophosphamide Induced Neutropenia

Administration of cyclophosphamide had caused a significant increase in the SGPT (Serum glutamic pyruvic transaminase) (p<0.001) level in cyclophosphamide control group when compared to placebo group. Administration of the commercial synthetic cinnamic acid (0.6 mg/tablet) low dose treated and the commercial synthetic cinnamic acid (6 mg/tablet) high dose treated to the animals caused a significant reversal (p<0.001) of SGPT (Serum glutamic pyruvic transaminase)_level when compared to the cyclophosphamide control group as tabulated in table 8B and FIG. 5B. The observed decrease in SGPT (Serum glutamic pyruvic transaminase) in the commercial synthetic cinnamic acid (0.6 mg/tablet) low dose treated and the commercial synthetic cinnamic acid (6 mg/tablet) high dose treated were comparable (p<0.001) to that of SGPT (Serum glutamic pyruvic transaminase) levels attained upon treatment with GCSF (granulocyte colony stimulating factor). However, this decrease was not superior to the decrease observed upon treatment with pure Picroside 1 and the degraded Picroside 1 without salt (cinnamic acid of the present disclosure).

Effect of the Commercial Synthetic Cinnamic Acid (0.6 mg/Tablet) Low Dose Treated and the Commercial Synthetic Cinnamic Acid (6 mg/Tablet) High Dose Treated on Serum Creatinine Level (mmol/L) in Cyclophosphamide Induced Neutropenia

The cyclophosphamide administration caused significantly (p<0.001) elevated level of creatinine when compared to the placebo group. A significant (p<0.001) decrease in creatinine level was observed following the administration of the commercial synthetic cinnamic acid (0.6 mg/tablet) low dose treated and the commercial synthetic cinnamic acid (6 mg/tablet) high dose treated compared to a cyclophosphamide control group as tabulated in table 8B and FIG. 5B. There was a significant (p<0.001) reduction in creatinine level of the GCSF treated group when compared to the cyclophosphamide control group. The decrease in creatinine level with the commercial synthetic cinnamic acid (0.6 mg/tablet) low dose treated and the commercial synthetic cinnamic acid (6 mg/tablet) high dose treated treatment was comparable and better than GCSF, pure Picroside 1 and the degraded Picroside 1 without salt (cinnamic acid of the present disclosure) treated groups.

Effect of the Commercial Synthetic Cinnamic Acid (0.6 mg/Tablet) Low Dose Treated and the Commercial Synthetic Cinnamic Acid (6 mg/Tablet) High Dose Treated on Blood Urea Nitrogen (mmol/L) in Cyclophosphamide Induced Nephrotoxicity.

The cyclophosphamide administration caused a significantly (p<0.001) elevated level of blood urea nitrogen (BUN) when compared to the placebo group. A significant (p<0.001) decrease in BUN level was seen following the administration of the commercial synthetic cinnamic acid (0.6 mg/tablet) low dose treated and the commercial synthetic cinnamic acid (6 mg/tablet) high dose treated compared to cyclophosphamide control group as tabulated in table 8B and FIG. 5B. There was a significant (p<0.001) decrease in BUN level of the GCSF (granulocyte colony stimulating factor) treated group when compared to the cyclophosphamide control group. The decrease in BUN level with the commercial synthetic cinnamic acid (0.6 mg/tablet) low dose treated and the commercial synthetic cinnamic acid (6 mg/tablet) high dose treated. GCSF (granulocyte colony stimulating factor), pure Picroside 1 and the degraded Picroside 1 without salt (cinnamic acid of the present disclosure) were similar in the respective groups.

Effect of the Commercial Synthetic Cinnamic Acid (0.6 mg/Tablet) Low Dose Treated and the Commercial Synthetic Cinnamic Acid (6 mg/Tablet) High Dose Treated on Uric Acid (mg/L) in Cyclophosphamide Induced Nephrotoxicity.

Administration of cyclophosphamide had caused a significant increase (p<0.001) in the serum uric acid (p<0.001) level in the cyclophosphamide control group. Administration of the commercial synthetic cinnamic acid (0.6 mg/tablet) low dose treated and the commercial synthetic cinnamic acid (6 mg/tablet) high dose treated to the animals caused a significant reversal (p<0.001) of uric acid level when compared to the cyclophosphamide control group as tabulated in table 8B and FIG. 5B. Treatment with GCSF (granulocyte colony stimulating factor) also caused a significant decrease in uric acid levels (p<0.001) when compared to cyclophosphamide control group. The decrease in serum uric acid level with the commercial synthetic cinnamic acid (0.6 mg/tablet) low dose treated and the commercial synthetic cinnamic acid (6 mg/tablet) high dose treated treatment was comparable and better than GCSF (granulocyte colony stimulating factor), pure Picroside 1 and the degraded Picroside 1 without salt (cinnamic acid of the present disclosure) treated groups.

TABLE 8B
The commercial synthetic cinnamic acid (0.6 mg/tablet) low dose treated and the commercial synthetic cinnamic
acid (6 mg/tablet) high dose treated on hepatic and renal parameters in Cyclophosphamide induced Neutropenia

| | | | | | The | | |
|---|---|---|---|---|---|---|---|
| | | | | | degraded | | |
| | | | | | Picroside | The | The |
| | | | G-CSF | | 1 without | commercial | commercial |
| | | | (granulocyte | | salt | synthetic | synthetic |
| | | | colony | | (cinnamic | Cinnamic | Cinnamic |
| | | CP | stimulating | Pure | acid of the | acid | acid |
| | Placebo | Control | factor) | Picroside | present | (0.6 mg/ | (6 mg/ |
| Parameters | control | (cyclophosphamide) | (2 μg) | 1 | disclosure) | tablet) | tablet) |
| SGOT | 20.5 ± | 136.7 ± | 32.17 ± | 25.67 ± | 27.5 ± | 33.67 ± | 29.33 ± |
| Serum | 1.432 | 2.963^{#} | 1.662^{$$,}*** | 0.5578*** | 2.766*** | 1.116^{#,}*** | 2.333^{$,}*** |
| glutamic | | | | | | | |
| oxaloacetic | | | | | | | |
| transaminase | | | | | | | |
| (IU/L) | | | | | | | |
| SGPT | 23 ± | 139.5 ± | 34.33 ± | 28.5 ± | 30.33 ± | 36.5 ± | 32.17 ± |
| (Serum | 1.732 | 2.861^{#} | 1.406^{$$,}*** | 0.6708*** | 2.753*** | 1.258^{#,}*** | 2.414^{$,}*** |
| glutamic | | | | | | | |
| pyruvic | | | | | | | |
| transaminase_(IU/L) | | | | | | | |
| Serum | 39.17 ± | 62.5 ± | 44.5 ± | 46.83 ± | 41 ± | 38.5 ± | 37.5 ± |
| Creatinine | 0.7923 | 0.7638^{#} | 0.8466^{$,}*** | 1.493^{$$,}*** | 2.145*** | 1.335*** | 1.176*** |
| (mmol/L) | | | | | | | |
| BUN (blood | 9.25 ± | 12.73 ± | 9.633 ± | 9.483 ± | 9.467 ± | 9.533 ± | 9.383 ± |
| urea | 0.07638 | 0.182^{#} | 0.08028*** | 0.08333*** | 0.08433*** | 0.09189*** | 0.1138*** |
| nitrogen) | | | | | | | |
| (mmol/L) | | | | | | | |
| Uric acid | 11.38 ± | 38.83 ± | 26.33 ± | 30.00 ± | 21.50± | 16.82 ± | 16.66 ± |
| (mg/L) | 0.4175 | 1.778^{#,}*** | 0.8819^{#,}*** | 0.6325^{#,}*** | 0.428^{#,}*** | 1.108^{$$,}*** | 1.144^{$$,}*** |

^{$}p < 0.05,
^{$$}p < 0.01,
^{#}p < 0.001 when compared to placebo group
*p < 0.01,
***p < 0.001 when compared to CP control

Inference: It is evident from table 8B that the test compounds (pure Picroside 1, the degraded Picroside without salt (cinnamic acid of the present disclosure), the commercial cinnamic acid (0.6 mg/tablet) low dose and the commercial cinnamic acid (6 mg/tablet) high dose were administered as 1 tablet per animal per day from day 5 for 5 days; CP control (cyclophosphamide) was administered with placebo tablet. Further, it is evident from table 8B that pure Picroside 1 and the degraded Picroside 1 without salt (cinnamic acid of the present disclosure) shows improved hepatic and renal parameters in neutropenic animal model as compared to the known anti-neutropenic protein recombinant drug GCSF (granulocyte colony stimulating factor).

### Experiment 11: Comparison of Lethal Dose of Cinnamic Acid of the Present Disclosure (Degraded Picroside 1 without Salt) and Dose Required for Efficacy for Alleviating Neutropenia

The lethal dose (LD50) of the commercial synthetic cinnamic acid was 5000 mg/kg while efficacy dose of the cinnamic acid was found to be 24 mg/kg.

The animal efficacy dose of the cinnamic acid prepared in accordance with the present disclosure was 0.6 mg/mice or 0.6 mg/25 g or 6 mg/250 g or 24 mg/kg mice.

The human dose of the cinnamic acid prepared in accordance with the present disclosure, AED (animal equivalent dose) was divided by 12.3=24/12.3=1.95 mg/kg HED (human equivalent dose).

For a 65 kg individual=60×1.95 mg=117 mg/dose

The dose regimen was administered for 5 days. Hence total HED (human equivalent dose) for a chemotherapy patient would be 117 mg×5=585 mg/dose taken by the cancer patients who had suffered from lower stages of cancer.

The lethal dose (LD50) of the commercial synthetic cinnamic acid was 5000 mg/kg in rats, while the efficacious dose of the cinnamic acid of the present disclosure (degraded Picroside 1 without salt) for the anti-neutropenic activity was found to be 24 mg/kg. Hence, it is evident that the efficacious animal dose of 24 mg/kg when administered to human equivalent dose of 2 mg/kg have no toxicity when given orally to neutropenic patients.

The animal efficacy dose of the cinnamic acid prepared in accordance with the present disclosure was 6 mg/25 g or 60 mg/250 g mice or 240 mg cinnamic acid/kg.

The human dose of the cinnamic acid prepared in accordance with the present disclosure, AED (animal equivalent dose) was divided by 12.3=240/12.3=19.51 mg/kg HED (human equivalent dose).

For a 65 kg individual=60×19.51 mg=1170 mg/dose

The dose regimen was administered for 5 days. Hence total HED (human equivalent dose) for a chemotherapy patient would be 1170 mg×5=5850 mg/dose taken by the cancer patients who had suffered from higher stages of cancer.

### Technical Advances and Economic Significance

The present disclosure described hereinabove has several technical advantages including, but not limited to, the realization of a pharmaceutical formulation and a process of preparing cinnamic acid, that
effectively improves the neutrophil count along with other blood cells without any serious adverse events of;
can be used in case of cyclophosphamide induced neutropenia;
improved patient compliance; and
can be suitably administered via oral, injectable, and inhalation route.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A pharmaceutical formulation comprising:
a) cinnamic acid as a pharmaceutically active agent in an amount in the range of 0.1 mass % to 10 mass % with respect to the total mass of the formulation; and
b) at least one excipient in an amount in the range of 90 mass % to 99.9 mass % with respect to the total mass of the formulation.

2. The pharmaceutical formulation as claimed in claim 1, wherein said excipient is at least one selected from the group consisting of solvent, colouring agent, lubricant, diluent and disintegrant.

3. The pharmaceutical formulation as claimed in claim 2, wherein said solvent is at least one selected from water, ethanol and isopropyl alcohol; said colouring agent is selected from Iron Oxide yellow NF; colour brilliant Blue supra; FD and C Green 3; and Sunset yellow; said lubricant is at least one selected from magnesium stearate, talc, silica, and stearic acid; said disintegrant is at least one selected from carboxymethylcellulose, and hydroxypropyl methylcellulose; and said diluent is selected from polyethylene glycol, dimethyl sulfoxide, ethyl lactate and a combination of D-Mannitol-Xylitol-Micro crystalline Cellulose-Crospovidone-Anhydrous dibasic calcium phosphate mixture.

4. The pharmaceutical formulation as claimed in claim 1, wherein said formulation is in a form selected from oral formulation, injectable formulation, dry powder inhalation formulation, metered dose-inhaler formulation, ointments, gels, patches, ophthalmic formulations, and sprays.

5. The pharmaceutical formulation as claimed in claim 4, wherein said oral formulation comprises cinnamic acid in an amount in the range of 0.1 mass % to 5 mass % with respect to the total mass of the formulation and at least one excipient in an amount in the range of 95 mass % to 99.9 mass % with respect to the total mass of the formulation, wherein said excipient is at least one selected from the group consisting of solvent, colouring agent, lubricant, diluent and disintegrant.

6. The pharmaceutical formulation as claimed in claim 5, wherein said solvent is at least one selected from water, ethanol and isopropyl alcohol; said colouring agent is selected from Iron Oxide yellow NF; colour brilliant Blue supra; FD and C Green 3; and Sunset yellow; said lubricant is at least one selected from magnesium stearate, talc, silica, and stearic acid; said disintegrant is at least one selected from carboxymethylcellulose, and hydroxypropyl methylcellulose; and said diluent is selected from polyethylene glycol, dimethyl sulfoxide, ethyl lactate and a combination of D-Mannitol-Xylitol-Micro crystalline Cellulose-Crospovidone-Anhydrous dibasic calcium phosphate mixture.

7. The pharmaceutical formulation as claimed in claim 4, wherein said injectable formulation comprises cinnamic acid in an amount in the range of 1 mass % to 10 mass % with respect to the total mass of the formulation and at least one excipient in an amount in the range of 90 mass % to 99 mass % with respect to the total mass of the formulation, is selected from the group consisting of polyethylene glycol, water, ethanol, propylene glycol, ethyl lactate and dimethyl sulfoxide.

8. The pharmaceutical formulation as claimed in claim 4, wherein said dry powder inhalation formulation comprises
a. micronized cinnamic acid having a particle size in the range of 0.1 μm to 10 μm, in an amount in the range of 1 mass % to 5 mass % with respect to the total mass of the formulation;
b. a first lactose having a particle size in the range of 20 μm to 300 μm, in an amount in the range of 10 mass % to 30 mass % with respect to the total mass of the formulation;
c. a second lactose having a particle size in the range of 0.1 μm to 10 μm, in an amount in the range of 75 mass % to 85 mass % with respect to the total mass of the formulation; and
d. an excipient in an amount in the range of 0.1 mass % to 1 mass % with respect to the total mass of the formulation,
wherein a mass ratio of said first lactose to said second lactose is in the range of 1:2 to 1:5.

9. The pharmaceutical formulation as claimed in claim 8, wherein said excipient is magnesium stearate.

10. The pharmaceutical formulation as claimed in claim 8, wherein the median mass aerodynamic diameter (MMAD) of said dry powder inhalation formulation is in the range of 2 μm to 5 μm.

11. The pharmaceutical formulation as claimed in claim 8, wherein the mean particle size (d10) of said first lactose is in the range of 35 μm to 65 μm; the mean particle size (d50) of said first lactose is in the range of 95 μm to 125 μm; and the mean particle size (d90) of said first lactose is in the range of 160 μm to 190 μm.

12. The pharmaceutical formulation as claimed in claim 8, wherein the mean particle size (d10) of said second lactose is in the range of 0.01 μm to 1 μm; the mean particle size (d50) of said second lactose is in the range of 1 μm to 5 μm; and the mean particle size (d90) of said second lactose is in the range of 1 μm to 10 μm.

13. The pharmaceutical formulation as claimed in claim 1, wherein said cinnamic acid is selected from trans-cinnamic acid and cis-cinnamic acid.

14. The pharmaceutical formulation as claimed in claim 1, wherein said cinnamic acid is administered at a dose in the range of 1.5 mg/kg body weight to 30 mg/kg body weight.

15. The pharmaceutical formulation as claimed in claim 1, wherein said formulation has an anti-neutropenic activity, anti-tuberculosis activity, anti-malarial activity, anti-viral activity against COVID-19, anti-bacterial activity and cardiovascular activity.

16. A method for treating neutropenia, tuberculosis, malaria, viral infection, bacterial infection and cardiovascular diseases in mammals, wherein said method comprises administering a mammal, a therapeutically effective amount of cinnamic acid in an amount in the range of 1.5 mg/kg body weight to 30 mg/kg body weight.

17. The method as claimed in claim 16, wherein said mammal is human.

18. The method as claimed in claim 16, wherein said therapeutically effective amount of cinnamic acid is in the range of 15 mg/kg body weight to 25 mg/kg body weight.

19. Use of the pharmaceutical formulation as claimed in claim 1, for the treatment of neutropenia, tuberculosis, malaria, viral infection, bacterial infection and cardiovascular diseases.

20. A process for the preparation of a cinnamic acid, said process comprising the following steps:
a) mixing Picroside 1 in a first fluid medium followed by adding at least one alkali hydroxide under stirring to obtain a mixture;
b) maintaining said mixture at a temperature in the range of 25° C. to 35° C. for a predetermined time period followed by neutralizing said mixture with hydrochloric acid to obtain a neutralized mixture;
c) removing said fluid medium from said neutralized mixture at a temperature in the range of 40° C. to 50° C. under vacuum to obtain a dry powder comprising a degraded Picroside 1 with salt;
d) mixing a second fluid medium to said dry powder to obtain a solution comprising a degraded Picroside 1 without salt; and
e) decanting said solution followed by evaporating said second fluid medium from said solution at a temperature in the range of 40° C. to 50° C. to obtain the cinnamic acid (the degraded Picroside 1 without salt).

21. The process as claimed in claim 20, wherein said first fluid medium is water and said second fluid medium is methanol, ethanol, propanol and butanol.

22. The process as claimed in claim 20, wherein said alkali hydroxide is sodium hydroxide, potassium hydroxide and calcium hydroxide.

23. The process as claimed in claim 20, wherein a ratio of said Picroside 1 to said alkali hydroxide is 0.01:50.

24. The process as claimed in claim 20, wherein said predetermined time period is in the range of 30 minutes to 2 hours.

25. Use of cinnamic acid as prepared according to claim 20, for the treatment of neutropenia, tuberculosis, malaria, viral infection, bacterial infection and cardiovascular diseases having a therapeutically effective amount in the range of 1.5 mg/kg body weight to 30 mg/kg body weight.

26. Use of cinnamic acid as prepared according to claim 20, for the treatment of neutropenia, tuberculosis, malaria, viral infection, bacterial infection and cardiovascular diseases, wherein said cinnamic acid is present in an amount in the range of 0.1 mass % to 10 mass % along with at least one excipient in an amount in the range of 90 mass % to 99.9 mass % with respect to the total mass of the formulation.
